SEQUENCE LISTING (2 sequences)

SEQ ID NO: 1 - DNA, 22 bp, Homo sapiens
gccagcgtgcaaataatgaagg

SEQ ID NO: 2 - DNA, 21 bp, Homo sapiens
agagggggattatgtatccac

(19) United States
(12) Patent Application Publication
(10) Pub. No.: US 2008/0161339 A1
(43) Pub. Date: Jul. 3, 2008

(54) Novel Thio-Containing Hydroxy-6-Phenylphenanthridines and their Use as Pde4 Inhibitors

(75) Inventor: Ulrich Kautz, Allensbach (DE)

Correspondence Address: NATH & ASSOCIATES PLLC, 112 South West Street, Alexandria, VA, 22314, US

(73) Assignee: ALTANA PHARMA AG, KONSTANZ (DE)

(21) Appl. No.: 10/591,768

(22) PCT Filed: Mar. 9, 2005
(86) PCT No.: PCT/EP2005/051052

§ 371 Date: Sep. 27, 2006

(30) Foreign Application Priority Data
Mar. 10, 2004 (EP) 04100988.7
Feb. 1, 2005 (EP) 05100692.2

(51) Int. Cl. A61K 31/473; C07D 221/12; A61P 11/00

(52) U.S. Cl. 514/298; 546/109

(57) ABSTRACT

Compounds of the formula I

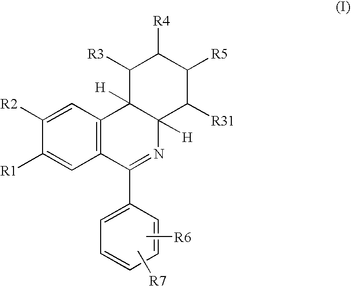

in which the substituents have the definitions provided in the specification, are novel, effective PDE4 inhibitors.

### FIELD OF APPLICATION OF THE INVENTION

The invention relates to novel thio-containing hydroxy-6-phenylphenanthridine derivatives, which are used in the pharmaceutical industry for the production of pharmaceutical compositions.

### KNOWN TECHNICAL BACKGROUND

The International Patent applications WO99/57118 and WO02/05616 describe 6-phenylphenanthridines as PDE4 inhibitors.

In the International Patent application WO99/05112 substituted 6-alkylphenanthridines are described as bronchial therapeutics.

In the European Patent application EP 0490823 dihydroisoquinoline derivatives are described which are useful in the treatment of asthma.

In the International Patent application WO9735854 phenanthridines substituted in 6-position are described as bronchial therapeutics.

In the International Patent application WO9905113 6-phenylphenanthridines are described as bronchial therapeutics.

In the International Patent application WO0042020 6-phenylphenanthridines are described as bronchial therapeutics.

### DESCRIPTION OF THE INVENTION

It has now been found that the novel thio-containing 2- or 3-hydroxy-6-phenylphenanthridines, which are described in greater detail below, differ from the previously known compounds by unanticipated and sophisticated structural alterations and have surprising and particularly advantageous properties.

The invention thus relates to compounds of formula I,

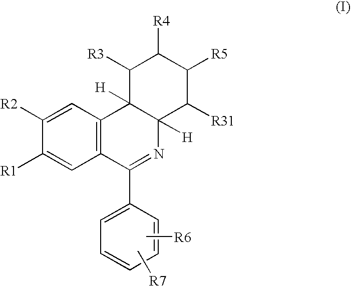

in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
   or in which
R1 and R2 together are a 1-2C-alkylenedioxy group,
R3 is hydrogen or 1-4C-alkyl,
R31 is hydrogen or 1-4C-alkyl,
   either, in a first embodiment (embodiment a) according to the present invention,
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
   or, in a second embodiment (embodiment b) according to the present invention,
R4 is hydrogen or 1-4C-alkyl, and
R5 is —O—R51, in which
R51 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl,
R6 is hydrogen, halogen, 1-4C-alkyl or 1-4C-alkoxy,
   in a first aspect (aspect 1) according to this invention,
R7 is —S(O)₂N(R8)R9, in which
R8 is hydrogen, 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
   or R8 and R9 together and with inclusion of the nitrogen atom, to which they are attached, form a heterocyclic ring Het1, in which
Het1 is optionally substituted by R81, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R81 is 1-4C-alkyl,
   or, in a second aspect (aspect 2) according to this invention,
R7 is -AN(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
   or, in a third aspect (aspect 3) according to this invention,
R7 is —S(O)ₙR12, in which
n is 0, 1 or 2,
R12 is 1-4C-alkyl,
   and the salts, the N-oxides and the salts of the N-oxides of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

2-4C-Alkyl represents a straight-chain or branched alkyl radical having 2 to 4 carbon atoms. Examples which may be mentioned are the butyl, isobutyl, sec-butyl, tertbutyl, propyl, isopropyl and preferably the ethyl radicals.

1-7C-Alkyl represents a straight-chain or branched alkyl radical having 1 to 7 carbon atoms. Examples which may be mentioned are the heptyl, isoheptyl (5-methylhexyl), hexyl, isohexyl (4-methylpentyl), neo-hexyl (3,3-dimethylbutyl), pentyl, isopentyl (3-methylbutyl), neopentyl (2,2-dimethylpropyl), butyl, isobutyl, sec-butyl, tertbutyl, propyl, isopropyl, ethyl or methyl radicals.

3-7C-Cycloalkyl represents cyclopropyl, cyclobutyl, cyclopentyl, cyclohexyl and cycloheptyl, of which cyclopropyl and cyclopentyl are preferred.

1-4C-Alkylene is a straight chain alkylene radical having 1 to 4 carbon atoms. Examples which may be mentioned in this context are the methylene (—CH₂—), ethylene (—CHᵣ—), trimethylene (—CH₂—CH₂—CH₂—) and the tetramethylene (—CH₂—CH₂—CH₂—CH₂—) radical.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

As completely or predominantly fluorine-substituted 1-4C-alkyl, for example, the 2,2,3,3,3-pentafluoropropyl, the perfluoroethyl, the 1,2,2-trifluoroethyl, in particular the 1,1,2,2-tetrafluoroethyl, the 2,2,2-trifluoroethyl, the trifluoromethyl and particularly the difluoromethyl radicals may be mentioned. “Predominantly” in this connection means that more than half of the hydrogen atoms of the 1-4C-alkyl radicals are replaced by fluorine atoms.

1-2C-Alkylenedioxy represents, for example, the methylenedioxy [—O—CH₂—O—] and the ethylenedioxy [—O—CH₂—CH₂—O—] radicals.

1-4C-Alkoxy-1-4C-alkyl represents one of the abovementioned 1-4C-alkyl radicals, which is substituted by one of the abovementioned 1-4C-alkoxy radicals. Examples which may be mentioned are the methoxymethyl, the methoxyethyl and the isopropoxyethyl radicals, particularly the 2-methoxyethyl and the 2-isopropoxyethyl radicals.

1-4C-alkoxy-2-4C-alkyl represents 2-4C-alkyl radicals, which are substituted by one of the abovementioned 1-4C-alkoxy radicals. Examples which may be mentioned are the methoxyethyl, ethoxyethyl and the isopropoxyethyl radicals, particularly the 2-methoxyethyl, 2-ethoxyethyl and the 2-isopropoxyethyl radicals.

1-7C-Alkylcarbonyl represents a radical which, in addition to the carbonyl group, contains one of the abovementioned 1-7C-alkyl radicals. Examples which may be mentioned are the acetyl, propionyl, butanoyl and hexanoyl radicals.

Hydroxy-2-4C-alkyl represents 2-4C-alkyl radicals, which are substituted by a hydroxyl group. Examples which may be mentioned are the 2-hydroxyethyl and the 3-hydroxypropyl radicals.

Halogen within the meaning of the invention is bromine, chlorine or fluorine.

When A has the meaning “bond”, then the moiety —N(R10)S(O)₂—R11 is directly attached to the phenyl radical.

Het1 is optionally substituted by R81, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur.

Het1 may include, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

As further examples for Het1 according to this invention may be mentioned, without being restricted thereto, R81 -substituted derivatives of the abovementioned exemplary Het1 radicals, notably, for example, Het1 radicals, which are substituted by R81 on a ring nitrogen atom, such as e.g. 4-N—(R81)-piperazinyl or 4-N—(R81 )-homopiperazinyl.

Illustratively, as an exemplary suitable Het1 radical may be mentioned, for example, without being restricted thereto, pyrrolidin-1-yl, morpholin-4-yl or 4-N—(R81)piperazin-1-yl, or piperidin-1-yl.

As it is known for the person skilled in the art, compounds comprising nitrogen atoms can form N-oxides. Particularly, imine nitrogen, especially heterocyclic or heteroaromatic imine nitrogen, or pyridine-type nitrogen (═N) atoms, can be N-oxidized to form the N-oxides comprising the group ═N⁺(O⁻)—. Thus, the compounds according to the present invention comprising the imine nitrogen atom in position 5 of the phenylphenanthridine backbone and, optionally (depending on the meaning of the substituents), one or more further nitrogen atoms suitable to exist in the N-oxide state (═W⁺(O⁻)—) may be capable to form (depending on the number of nitrogen atoms suitable to form stabile N-oxides) mono-oxides, bis-N-oxides or multi-N-oxides, or mixtures thereof.

The term N-oxide(s) as used in this invention therefore encompasses all possible, and in particular all stabile, N-oxide forms, such as mono-N-oxides, bis-N-oxides or multi-N-oxides, or mixtures thereof in any mixing ratio.

Possible salts for compounds of the formula I—depending on substitution— are all acid addition salts or all sails with bases. Particular mention may be made of the pharmacologically tolerable sails of the inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, it being possible to employ the acids in salt preparation—depending on whether a mono- or polybasic acid is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person skilled in the art.

It is known to the person skilled in the art that the compounds according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds of the formula I.

The substituents R6 and R7 of compounds of formula I can be attached in the ortho, meta or para position with respect to the binding position in which the 6-phenyl ring is bonded to the phenanthridine ring system, whereby preference is given to the attachement in the meta or in the para position. In one embodiment R6 is hydrogen or methyl, and the radical R7 is attached in the meta or in the para position with respect to the binding position in which the 6-phenyl ring is bonded to the phenanthridine ring system. In a particular embodiment R6 is hydrogen, and the radical R7 is attached in the meta or in the para position.

Compounds of formula I to be more worthy to be mentioned are those in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely predominantly fluorine-substituted 1-2C-alkoxy,
R3 is hydrogen,
R31 is hydrogen,
   either, in a first embodiment (embodiment a) according to the present invention,
R4 is —O—R41, in which
R41 is hydrogen or 1-7C-alkylcarbonyl,
R5 is hydrogen,
   or, in a second embodiment (embodiment b) according to the present invention,
R4 is hydrogen, and
R5 is —O—R51, in which
R51 is hydrogen or 1-7C-alkylcarbonyl,
R6 is hydrogen,
   in a first aspect (aspect 1) according to this invention,
R7 is —S(O)₂N(R8)R9, in which
R8 is hydrogen, 1-4C-alkyl, 1-4C-alkoxy-2-4C-alkyl or 3-7C-cycloalkyl,
R9 is hydrogen, 1-4C-alkyl or 1-4C-alkoxy-2-4 C-alkyl,
   or R8 and R9 together and with inclusion of the nitrogen atom, to which they are attached, form a heterocyclic ring Het1, in which
Het1 is optionally substituted by R81, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R81 is 1-4C-alkyl,
   or, in a second aspect (aspect 2) according to this invention,
R7 is -A-N(R10)S(O)₂—R11, in which
A is a bond or 1-4C-alkylene,
R10 is hydrogen or 1-4C-alkyl,
R11 is 1-4C-alkyl, or R 111-substituted phenyl, in which
R111 is halogen or 1-4C-alkyl,
   or, in a third aspect (aspect 3) according to this invention,
R7 is —S(O)ₙR12, in which
n is 0, 1 or 2,
R12 is 1-4C-alkyl,
   and the salts, the N-oxides and the salts of the N-oxides of these compounds.

Yet compounds of formula I to be more worthy to be mentioned are those in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely predominantly fluorine-substituted 1-2C-alkoxy,
R3 is hydrogen,
R31 is hydrogen,
   either, in a first embodiment (embodiment a) according to the present invention,
R4 is —O—R41, in which
R41 is hydrogen or 1-7C-alkylcarbonyl,
R5 is hydrogen,
   or, in a second embodiment (embodiment b) according to the present invention,
R4 is hydrogen, and
R5 is —O—R51, in which
R51 is hydrogen or 1-7C-alkylcarbonyl,
R6 is hydrogen or 1-4C-alkyl,
   in a first aspect (aspect 1) according to this invention,
R7 is —S(O)₂N(R8)R9, in which
R8 is hydrogen, 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
   or R8 and R9 together and with inclusion of the nitrogen atom, to which they are attached, form a heterocyclic ring Het1, in which
Het1 is a 3- to 7-membered saturated monocyclic heterocyclic ring radical comprising the nitrogen atom, to which R8 and R9 are bonded, and optionally one further heteroatom selected from the group consisting of oxygen, nitrogen, N(R81) and sulfur, in which
R81 is 1-4C-alkyl,
   or, in a second aspect (aspect 2) according to this invention,
R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
   or, in a third aspect (aspect 3) according to this invention,
R7 is —S(O)ₙR12, in which
n is 0, 1 or 2,
R12 is 1-4C-alkyl,
   and the salts, the N-oxides and the salts of the N-oxides of these compounds.

Compounds of formula I in particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is 1-4C-alkylcarbonyl or, in particular, in an individual embodiment according to this invention, hydrogen,
R5 is hydrogen,
R6 is hydrogen,
   in a first aspect (aspect 1) according to this invention,
R7 is —S(O)₂N(R8)R9, in which
R8 is 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
   or R8 and R9 together and with inclusion of the nitrogen atom, to which they are attached, form a heterocyclic ring Het1, in which
Het1 is morpholinyl, thiomorpholinyl, pyrrolidinyl, 4N-(R81)-piperazinyl, 4N-(R81)-homopiperazinyl, in which
R81 is 1-4C-alkyl,
   or, in a second aspect (aspect 2) according to this invention,
   R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
   or, in a third aspect (aspect 3) according to this invention,
R7 is —S(O)ₙR12, in which
n is 0 or 2,
R12 is 1-4C-alkyl,
   and the salts, the N-oxides and the salts of the N-oxides of these compounds.

Yet compounds of formula I in particular worthy to be mentioned are those in which

R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is 1-4C-alkylcarbonyl or, in particular, in an individual embodiment according to this invention, hydrogen,
R5 is hydrogen,
R6 is hydrogen or methyl,
   in a first aspect (aspect 1) according to this invention,
R7 is —S(O)₂N(R8)R9, in which
R8 is 1-4C-alkyl, 1-4C-alkoxy-2-4C-alkyl or 3-7C-cycloalkyl,
R9 is hydrogen, 1-4alkyl or 1-4-alkoxy-2-4alkyl,
   or R8 and R9 together and with inclusion of the nitrogen atom, to which they are attached, form a heterocyclic ring Het1, in which
Het1 is morpholinyl, thiomorpholinyl, pyrrolidinyl, piperidinyl, 4-N—(R81)piperazinyl, or 4-N—(R81)-homopiperazinyl, in which

### R81 is 1-4C-alkyl,

or, in a second aspect (aspect 2) according to this invention,
R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
   or, in a third aspect (aspect 3) according to this invention,
R7 is —S(O)ₙR12, in which
n is 0, 1 or 2,
R12 is 1-4C-alkyl,
   and the salts, the N-oxides and the salts of the N-oxides of these compounds.

Compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
   in a first aspect (aspect 1) according to this invention,
R7 is —S(O)₂N(R8)R9, in which
R8 is 1-4C-alkyl, 1-4C-alkoxy-ethyl or 3-5C-cycloalkyl,
   R9 is hydrogen, 1-4C-alkyl or 1-4C-alkoxy-ethyl,
   or R8 and R9 together and with inclusion of the nitrogen atom, to which they are attached, form a heterocyclic ring Het1, in which
   Het1 is morpholinyl, pyrrolidinyl or 4-N—(R81)-piperazinyl, in which
R81 is 1-4C-alkyl,
   or, in a second aspect (aspect 2) according to this invention,
R7 is -A-N(R10)S(O)₂—R11, in which
A is a bond or 1-2C-alkylene,
R10 is hydrogen or 1-4C-alkyl,
R11 is 1-4C-alkyl, or R11-substituted phenyl, in which
R111 is fluorine or 1-4C-alkyl,
   or, in a third aspect (aspect 3) according to this invention,
R7 is —S(O)ₙR12, in which
n is 0 or 2,
R12 is 1-4C-alkyl,
   and the salts, the N-oxides and the salts of the N-oxides of these compounds.

Yet compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is acetyl or, particularly, hydrogen,
R5 is hydrogen,
R6 is hydrogen or methyl,
   in a first aspect (aspect 1) according to this invention,
R7 is —S(O)₂—N(R8)R9, in which
R8 is 1-4C-alkyl, 1-4C-alkoxy-ethyl or 3-5C-cycloalkyl,
   R9 is hydrogen, 1-4C-alkyl or 1-4C-alkoxy-ethyl,
   or R8 and R9 together and with inclusion of the nitrogen atom, to which they are attached, form a heterocyclic ring Het1, in which
   Het1 is morpholinyl, pyrrolidinyl, piperidinyl or 4N—(R81)-piperazinyl, in which
R81 is 1-4C-alkyl,
   or, in a second aspect (aspect 2) according to this invention,
R7 is -A-N(R10)S(O)₂—R11, in which
A is a bond or 1-2C-alkylene,
R10 is hydrogen or 1-4C-alkyl,
R11 is 1-4C-alkyl, or R111-substituted phenyl, in which
R111 is fluorine, chlorine or 1-4C-alkyl,
   or, in a third aspect (aspect 3) according to this invention,
R7 is —S(O)ₙR12, in which
n is 0, 1 or 2,
R12 is 1-4C-alkyl,
   and the salts, the N-oxides and the salts of the N-oxides of these compounds.

Compounds of formula I in further more particular worthy to be mentioned are those in which
R1 is methoxy or ethoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
   in a first aspect (aspect 1) according to this invention,
R7 is —S(O)₂N(R8)R9, in which
R8 is methyl, ethyl, propyl, 2-methoxy-ethyl or cyclopropyl,
R9 is hydrogen, methyl, ethyl, propyl or 2-methoxy-ethyl,
   or R8 and R9 together and with inclusion of the nitrogen atom, to which they are attached, form a heterocyclic ring Het1, in which
Het1 is morpholinyl, pyrrolidinyl, piperidinyl or 4-N—R81)-piperazinyl, in which
R81 is methyl,
   or, in a second aspect (aspect 2) according to this invention,
R7 is -A-N(R10)S(O)₂—R11, in which
A is a bond or methylene,
R10 is hydrogen or methyl,
R11 is R111-substituted phenyl, in which
R111 is fluorine, chlorine or methyl,
   or, in a third aspect (aspect 3) according to this invention,
R7 is —S(O)ₙR12, in which
n is 0, 1 or 2,
R12 is 1-4C-alkyl, such as e.g. methyl,
   and the salts, the N-oxides and the salts of the N-oxides of these compounds.

A particular embodiment of compounds of formula I in further more particular worthy to be mentioned include those compounds of formula I in which
R1 is methoxy,
R2 is ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
   in a first aspect (aspect 1) according to this invention,
R7 is —S(O)₂N(R8)R9, in which
R8 is methyl, ethyl, propyl, 2-methoxy-ethyl or cyclopropyl,
R9 is hydrogen, methyl, ethyl, propyl or 2-methoxy-ethyl,
   or R8 and R9 together and with inclusion of the nitrogen atom, to which they are attached, form a heterocyclic ring Het1, in which
Het1 is morpholinyl, pyrrolidinyl, piperidinyl or 4-N—(R81)-piperazinyl, in which
R81 is methyl,
   or, in a second aspect (aspect 2) according to this invention,
R7 is -A-N(R10)S(O)₂—R11, in which
A is a bond or methylene,
R10 is hydrogen or methyl,
R11 is R111-substituted phenyl, in which
R111 is fluorine, chlorine or methyl, such as, for example, 4-(R111)-phenyl, e.g. 4-methylphenyl or 4-fluorophenyl,
   or, in a third aspect (aspect 3) according to this invention,
R7 is —S(O)ₙR12, in which
n is 0, 1 or 2,
R12 is 1-4C-alkyl, such as e.g. methyl,
   and the salts, the N-oxides and the salts of the N-oxides of these compounds.

Compounds of formula I in yet further more particular worthy to be mentioned are those in which
R1 is methoxy or ethoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —S(O)ₙR12, in which
n is 0 or 1,
R12 is methyl,
   and the salts, the N-oxides and the salts of the N-oxides of these compounds.

Yet compounds of formula I in yet further more particular worthy to be mentioned are those in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is bonded to the meta or para position with respect to the binding position in which the 6-phenyl ring is bonded to the phenanthridine ring system, and is —S(O)ₙR12, in which
n is0, 1 or 2,
R12 is 1-4C-alkyl, such as e.g. methyl,
   and the salts, the N-oxides and the salts of the N-oxides of these compounds.

A particular embodiment of compounds of formula I in yet further more particular worthy to be mentioned include those compounds of formula I in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is bonded to the para position with respect to the binding position in which the 6-phenyl ring is bonded to the phenanthridine ring system, and is —S(O)ₙR12, in which
n is 0,
R12 is methyl,
   and the salts, the N-oxides and the salts of the N-oxides of these compounds.

Yet a particular embodiment of compounds of formula I in yet further more particular worthy to be mentioned include those compounds of formula I in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is bonded to the para position with respect to the binding position in which the 6-phenyl ring is bonded to the phenanthridine ring system, and is —S(O)ₙR12, in which
n is 1,
R12 is methyl,
   and the salts, the N-oxides and the salts of the N-oxides of these compounds.

Still yet a particular embodiment of compounds of formula I in yet further more particular worthy to be mentioned include those compounds of formula I in which
R1 is methoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is bonded to the para position with respect to the binding position in which the 6phenyl ring is bonded to the phenanthridine ring system, and is —S(O)ₙR12, in which
n is2,
R12 is methyl,
   and the salts, the N-oxides and the salts of the N-oxides of these compounds.

A special interest in the compounds according to this invention relates to those compounds which are included—within the meaning of the present invention—by one or, when possible, by more of the following embodiments:

A special embodiment of the compounds of the present invention include those compounds of formula I in which R1 and R2 are independently 1-2C-alkoxy, 2,2-difluoroethoxy, or completely or predominantly fluorine-substituted 1-2C-alkoxy.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3 and R31 are both hydrogen.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3, R31 and R6 are all hydrogen.

Another special embodiment of the compounds of the present invention include those compounds of formula I in which one of R1 and R2 is methoxy, and the other is methoxy, ethoxy, difluoromethoxy or 2,2-difluoroethoxy, and R3 and R31 are both hydrogen.

Another special embodiment of the compounds of the present invention include those compounds of formula I in which R1 is ethoxy or, particularly, methoxy, and R2 is methoxy, or, particularly, ethoxy, difluoromethoxy or 2,2-difluoroethoxy, and R3 and R31 are both hydrogen.

Another special embodiment of the compounds of the present invention include those compounds of formula I in which R1 is methoxy, and R2 is methoxy, ethoxy, difluoromethoxy or 2,2-difluoroethoxy, and R3 and R31 are both hydrogen.

Another special embodiment of the compounds of the present invention include those compounds of formula I in which R1 is methoxy, and R2 is ethoxy, difluoromethoxy or 2,2-fluoroethoxy, and R3 and R31 are both hydrogen.

Another special embodiment of the compounds of the present invention include those compounds of formula I in which one of R1 and R2 is 2,2-difluoroethoxy, and the other is different from 2,2-difluoroethoxy, and R3 and R31 are both hydrogen.

Another special embodiment of the compounds of the present invention include those compounds of formula I in which R1 is ethoxy or, particularly, methoxy, and R2 is 2,2-difluoroethoxy, and R3 and R31 are both hydrogen.

Another special embodiment of the compounds of the present invention include those compounds of formula I in which R1 is methoxy, and R2 is 2,2-difluoroethoxy, and R3 and R31 are both hydrogen.

Another special embodiment of the compounds of the present invention include those compounds of formula I in which R1 is methoxy, and R2 is ethoxy, and R3 and R31 are both hydrogen.

Another special embodiment of the compounds of the present invention include those compounds of formula I in which R1 is methoxy, and R2 is difluoromethoxy, and R3 and R31 are both hydrogen.

Another special embodiment of the compounds of the present invention include those compounds of formula 1, in which R5 or, particularly, R4 is the radical (1-4C-alkylcarbonyl)-O— such as e.g. acetoxy, or hydroxyl, and all the other substituents are as defined in any compound mentioned above.

Another special embodiment of the compounds of the present invention include those compounds of formula I in which R5 or, particularly, R4 is hydroxyl.

Another special embodiment of the compounds of the present invention include those compounds of formula I in which R6 is hydrogen or methyl.

Another special embodiment of the compounds of the present invention include those compounds of formula I according to aspect 1.

Another special embodiment of the compounds of the present invention include those compounds of formula I according to aspect 2.

Another special embodiment of the compounds of the present invention include those compounds of formula I according to aspect 3.

Another special embodiment of the compounds of the present invention include those compounds of formula I according to aspect 3 of this invention in which R7 is —S(O)ₙ—R12, in which
R12 is 1-4C-alkyl, such as e.g. methyl, and
n is 0 or 1.

Another special embodiment of the compounds of the present invention include those compounds of formula I according to aspect 3 of this invention in which R7 is —S(O)ₙ—R12, in which
R12 is 1-4C-alkyl, such as e.g. methyl, and
n is 1.

A preferred embodiment according to the present invention is embodiment a.

A further preferred embodiment of the compounds of the present invention include compounds according to embodiment a, in which R5 and R41 are both hydrogen, and in which R1 and R2 are independently 1-2C-alkoxy, 2,2-difluoroethoxy, or completely or predominantly fluorine-substituted 1-2C-alkoxy, and R3, R31 and R6 are all hydrogen.

A yet further preferred embodiment of the compounds of the present invention include compounds according to embodiment a, in which R5 is hydrogen, and in which R1 is methoxy, and R2 is ethoxy, difluoromethoxy or 2,2-difluoroethoxy, and R3 and R31 are both hydrogen.

A still yet further preferred embodiment of the compounds of the present invention include compounds according to embodiment a, in which R5 and R41 are both hydrogen, and in which R1 is methoxy, and R2 is ethoxy, difluoromethoxy or 2,2-difluoroethoxy, and R3 and R31 are both hydrogen.

Suitable compounds according to the present invention more worthy to be mentioned include those compounds of formula I, in which R5 or, particularly, R4 is hydroxyl.

Exemplary compounds according to the present invention may include, without being restricted thereto, compounds selected from the group consisting of those compounds mentioned in the following examples as final compounds, particularly those examples which are from formula I according to embodiment a, in which R3, R31, R41 and R5 are all hydrogen, the enantiomers, as well as the salts, the N-oxides and the salts of the N-oxides of these compounds and enantiomers.

Preferably, the compounds according to the present invention which are listed in the Table A in the appended “Biological Investigations” and, particularly, the enantiomers thereof, particularly those having the formula Ia*****, as well as the salts of these compounds and enantiomers, are to be mentioned as a particular interesting aspect of the present invention.

The compounds of formula I are chiral compounds having chiral centers at least in positions 4a and 10b and depending on the meanings of R3, R31, R4 and R5 additional chiral centers in positions 1, 2, 3 and 4.

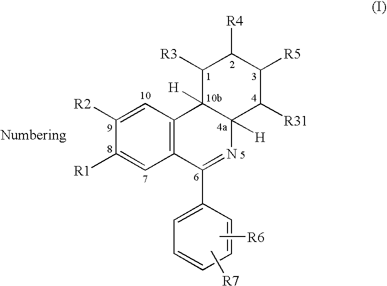

The invention includes all conceivable stereoisomers in pure form as well as in any mixing ratio. Preference is given to compounds of formula I in which the hydrogen atoms in positions 4a and 10b are in the cis position relative to one another. The pure cis enantiomers and their mixtures in any mixing ratio and including the racemates are more preferred in this context.

Particularly preferred in this context are those compounds of formula 1, which have with respect to the positions 4a and 10b the configuration shown in formula (I*):

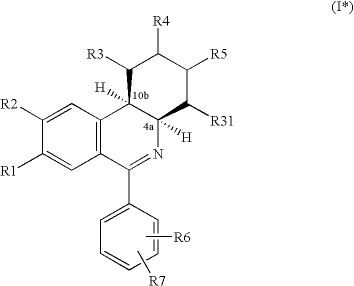

If, for example, in compounds of formula I* R3, R31 and R5 have the meaning hydrogen and R4 has the meaning —OR41, then the configuration—according to the rules of Cahn, Ingold and Prelog—is R in the 4a position and R in the 10b position.

Preferred compounds of the formula I according to embodiment a are those which have, with respect to the positions 2,4a and 10b, the same configuration as shown in the formulae Ia** and Ia*** and Ia****:

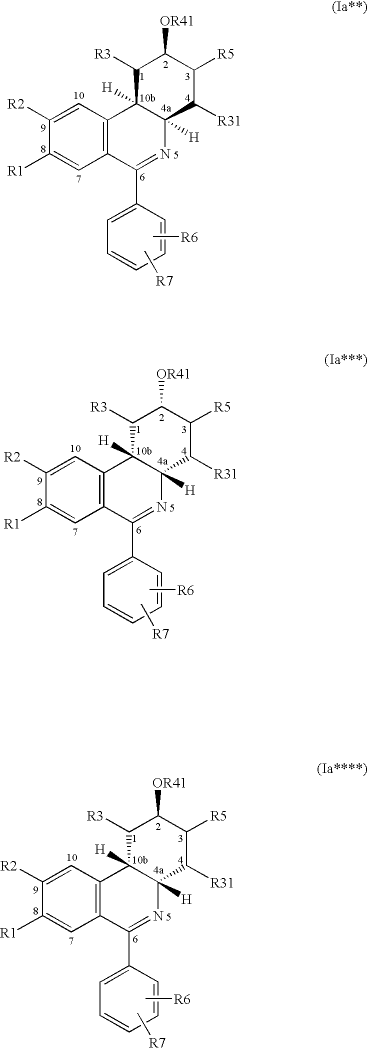

If, for example in compounds of the formula Ia** R3, R31 and R5 have the meaning hydrogen, then the configuration—according the rules of Cahn, Ingold and Prelog—is S in the position 2, R in the position 4a and R in the position 10b.

If, for example in compounds of the formula Ia*** R3, R31 and R5 have the meaning hydrogen, then the configuration—according the rules of Cahn, Ingold and Prelog—is R in the position 2, S in the position 4a and S in the position 10b.

If, for example in compounds of the formula Ia**** R3, R31 and R5 have the meaning hydrogen, then the configuration—according the rules of Cahn, Ingold and Prelog—is S in the position 2, S in the position 4a and S in the position 10b.

In more particular preferred compounds of the formula I according embodiment a are those which have, with respect to the positions 2, 4a and 10b, the same configuration as shown in the formula Ia*****:

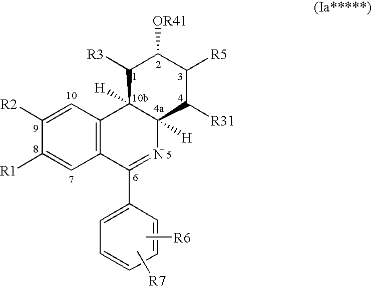

If, for example in compounds of the formula Ia***** R3, R31 and R5 have the meaning hydrogen, then the configuration—according the rules of Cahn, Ingold and Prelog—is R in the position 2, R in the position 4a and R in the position 10b.

Preferred compounds of the formula I according to embodiment b are those which have, with respect to the positions 3, 4a and 10b, the same configuration as shown in the formulae Ib** and Ib*** and Ib****:

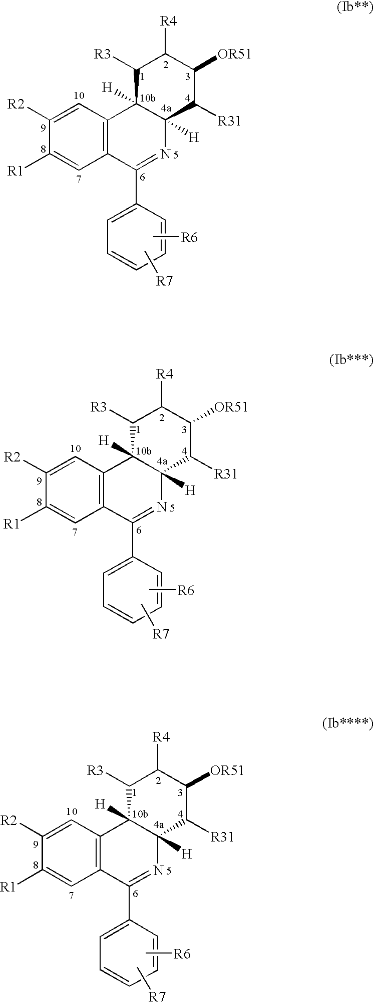

If, for example in compounds of the formula Ib** R3, R31 and R5 have the meaning hydrogen, then the configuration—according the rules of Cahn, Ingold and Prelog—is R in the position 3, R in the position 4a and R in the position 10b.

If, for example in compounds of the formula Ib*** R3, R31 and R5 have the meaning hydrogen, then the configuration—according the rules of Cahn, Ingold and Prelog—is S in the position 3, S in the position 4a and S in the position 10b.

If, for example in compounds of the formula Ib**** R3, R31 and R5 have the meaning hydrogen, then the configuration—according the rules of Cahn, Ingold and Prelog—is R in the position 3, S in the position 4a and S in the position 10b.

In more particular preferred compounds of the formula I according to embodiment b are those which have, with respect to the positions 3, 4a and 10b, the same configuration as shown in the formula Ib*****:

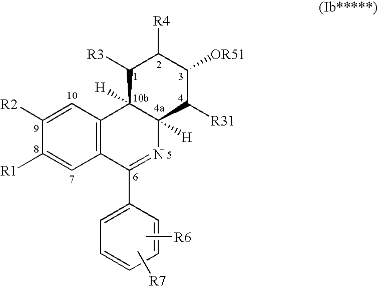

If, for example in compounds of the formula Ib***** R3, R31 and R5 have the meaning hydrogen, then the configuration—according the rules of Cahn, Ingold and Prelog—is S in the position 3, R in the position 4a and R in the position 10b.

Within the meaning of the embodiments a and b according this invention, compounds of formula Ia***** are in particular to be emphasized.

The enantiomers can be separated in a manner known per se (for example by preparation and separation of appropriate diastereoisomeric compounds). Preferably, an enantiomer separation is carried out at the stage of the starting compounds having a free amino group such as starting compounds of formulae IVa, in which R1, R2, R3, R31, R41 and R5 have the meanings given above, or VIIb as defined below.

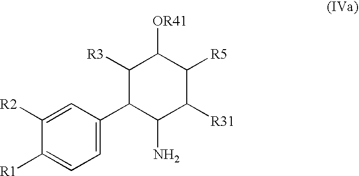

Separation of the enantiomers can be carried out, for example, by means of salt formation of the racemic compounds of the formulae IVa or VIIb with optically active acids, preferably carboxylic acids, subsequent resolution of the salts and release of the desired compound from the salt. Examples of optically active carboxylic acids which may be mentioned in this connection are the enantiomeric forms of mandelic acid, tartaric acid, O,O′-dibenzoyltartaric acid, camphoric acid, quinic acid, glutamic acid, pyroglutamic acid, malls acid, camphorsulfonic acid, 3-bromocamphorsulfonic acid, α-methoxyphenylacetic acid, (α-methoxy-α-trifluoromethylphenylacetic acid and 2phenylpropionic acid. Alternatively, enantiomerically pure starting compounds of the formulae can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystallization from a suitable solvent.

The compounds according to the invention can be prepared, for example, as shown in the reaction schemes below and according to the following specified reaction steps, or, particularly, in a manner as described by way of example in the following examples, or analogously or similarly thereto according to preparation procedures or synthesis strategies known to the person skilled in the art.

Compounds of formula I, in which R1, R2, R3, R31, R4, R5, R6 and R7 have the meanings mentioned above, according to embodiment a or b (i.e. compounds of formulae Ia or Ib, respectively) can be obtained as described as follows.

Compounds of formula Ia according to embodiment a can be prepared as described and shown in reaction scheme 1 below.

In the first reaction step of the synthesis route shown in scheme 1, compounds of the formula Va, in which R1, R2, R3, R31, R41 and R5 have the meanings mentioned above in embodiment a whereby R41 is other than hydrogen, are prepared from the corresponding compounds of the formula VIa by introduction of the group R41, which is other than hydrogen. The introduction reaction is carried out in a manner habitual per se for an etherification or esterification reaction, or as described by way of example in the following examples.

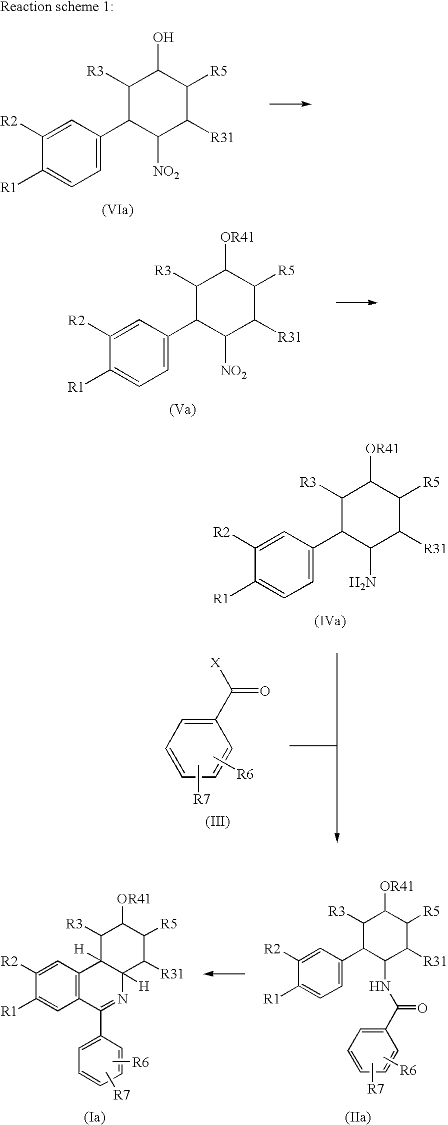

In the next reaction step of the synthesis route shown in reaction scheme 1, the nitro group of compounds of the formula Va, in which R1, R2, R3, R31, R41 and R5 have the meanings mentioned above in embodiment a whereby R41 is other than hydrogen, is reduced to the amino group of the corresponding compounds of the formula IVa. Said reduction is carried out in a manner known to the person skilled in the art, for example as described in J. Org. Chem. 1962, 27, 4426 or as described in the following examples. In more detail, the reduction can be carried out, for example, by catalytic hydrogenation, e.g. in the presence of Raney nickel or a noble metal catalyst such as palladium on active carbon, in a suitable solvent such as methanol or ethanol at room temperature and under normal or elevated pressure. Optionally, a catalytic amount of an acid, such as, for example, hydrochloric acid, can be added to the solvent. Preferably, however, the reduction is carried out using a hydrogen-producing mixture, for example, metals such as zinc, zinc-copper couple or iron with organic acids such as acetic acid or mineral acids such as hydrochloric acid. More preferably, the reduction is carried out using a zinc-copper couple in the presence of an organic or an inorganic acid. Such a zinc-copper couple is accessible in a way known to the person of ordinary skill in the art.

Compounds of the formula IVa, in which R1, R2, R3, R31, R41 and R5 have the meanings indicated above in embodiment a whereby R41 is other than hydrogen and which are sensitive against catalytic hydrogenation, can be prepared from the corresponding compounds of the formula Va by selective reduction of the nitro group in a manner known to the person skilled in the art, for example by hydrogen transfer reaction in the presence of a metal catalyst, for example palladium or, preferably, Raney nickel, in a lower alcohol as solvent using, for example, ammonium formiate or, preferably, hydrazine hydrate as hydrogen donor.

Compounds of the formula IIa, in which R1, R2, R3, R31, R41, R5, R6 and R7 have the meanings indicated above in embodiment a whereby R41 is other than hydrogen, are accessible from the corresponding compounds of the formula IVa by reaction with corresponding compounds of the formula III, in which X represents a suitable leaving group, preferably a chlorine atom.

Alternatively, compounds of the formula IIa can also be prepared from the corresponding compounds of the formula IVa and corresponding compounds of the formula III, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)N,N,N′,N′-tetramethyluronium tetrafluoroborate or O-(benzotriazol-1yl)N,N,N′,N′-tetramthyluronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

Compounds of the formula III are either known or can be prepared in a known manner.

Compounds of the formula Ia, in which R1, R2, R3, R31, R41, R5, R6 and R7 have the meanings mentioned in embodiment a whereby R41 is other than hydrogen, can be obtained by cyclocondensation of corresponding compounds of the formula IIa.

Said cyclocondensation reaction is carried out in a manner known per se to the person skilled in the art or as described by way of example in the following examples, according to Bischler-Napieralski (e.g. as described in J. Chem. Soc., 1956, 42804282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as isopropyl acetate or acetonitrile, or without further solvent using an excess of condensing agent, at reduced temperature, or at room temperature, or at elevated temperature or at the boiling temperature of the solvent or condensing agent used. If necessary, said cyclocondensation reaction can be carried out in the presence of one or more suitable Lewis Acids such as, for example, suitable metal halogenides (e.g. chlorides) or sulphonates (e.g. triflates), including rare earth metal salts, such as e.g. anhydrous aluminum trichloride, aluminum tribromide, zinc chloride, boron trifluoride ethereate, titanium tetrachloride or, in particular, tin tetrachloride, and the like.

Below reaction scheme 2 shows the synthesis of compounds of the formula VIa, in which R1, R2, R3, R31 and R5 have the meanings indicated above in embodiment a, from corresponding compounds of the formula VIIa via reduction reaction of the carbonyl group. Suitable reducing agents for the abovementioned reduction reaction may include, for example, metal hydride compounds such as, for example, diisopropylaluminium hydride, borane, sodium borohydride, sodium triacetoxyborohydride, sodium cyanoborohydride, zinc borohydride, potassium tri-sec-butylborohydride, sodium tri-sec-butylborohydride, lithium tri-sec-butylborohydride, β-isopinocampheyl-9-borabicyclo[3.3.1]nonane and the like. The preferred examples of said reducing agents are sodium cyanoborohydride, β-isopinocampheyl-9-borabicyclo[3.3.1]nonane and potassium tri-sec-butylborohydride. The most preferred examples of the abovementioned reducing agents are β-isopinocampheyl-9-borabicyclo[3.3.1]nonane and potassium tri-sec-butylborohydride, which both allow to prepare compounds of the formula VIa stereoselectively. “Stereoselectively” in this connection means that those compounds of the formula VIa, in which the hydrogen atoms in positions 1 and 3 are located at the opposite side of the plane defined by the cyclohexane ring, are obtained preferentially.

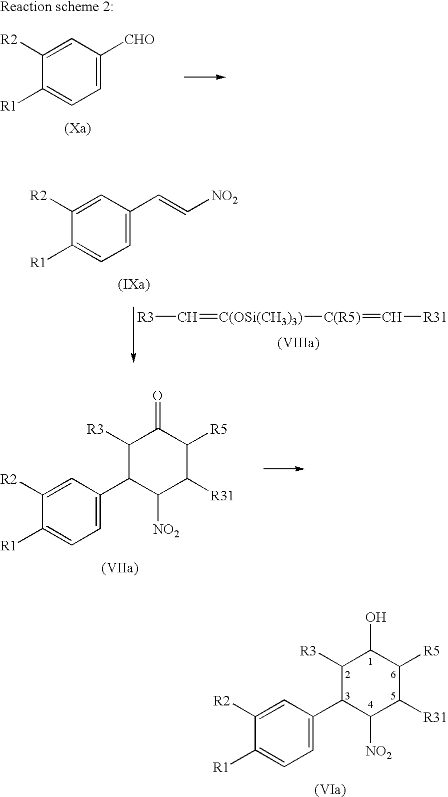

The compounds of the formula VIIa, in which R1, R2, R3, R31 and R5 have the meanings mentioned in embodiment a, are either known or can be obtained by the reaction of compounds of the formula IXa, in which R1 and R2 have the meanings mentioned above, with compounds of the formula VIIIa, in which R3, R31 and R5 have the meanings mentioned above in embodiment a. The cycloaddition reaction is carried out in a manner known to the person skilled in the art according to Diels-Alder, e.g. as described in J. Amer. Chem. Soc. 1957, 79, 6559 or in J. Org. Chem. 1952, 17, 581 or as described in the following examples.

Compounds of the formulae VIa or Va, in which the phenyl ring and the nitro group are trans to one another, can be converted in a manner known to the person skilled in the art into the corresponding cis compounds, e.g. as described in J. Amer. Chem. Soc. 1957, 79, 6559 or as described in the following examples.

The compounds of the formulae VIIIa and IXa are either known or can be prepared in a known manner. The compounds of the formula IXa can be prepared, for example, in a manner known to the person skilled in the art from corresponding compounds of the formula Xa as described, for example, in J. Chem. Soc. 1951, 2524 or in J. Org. Chem. 1944, 9, 170 or as described in the following examples.

The compounds of the formula Xa, in which R1 and R2 have the meanings indicated above in embodiment a, are either known or can be prepared in a manner known to the person skilled in the art, as described, for example, in Ber. Dtsch. Chem. Ges. 1925, 58, 203.

Compounds of formula Ib according to embodiment b, in which R1, R2, R3, R31, R4 and R51 have the meanings indicated above in embodiment b whereby R51 is other than hydrogen, can be prepared as described and shown in reaction scheme 3 below.

In the first reaction step in reaction scheme 3, the nitro group of compounds of the formula VIIIb, in which R1, R2, R3, R31 and R4 have the meanings indicated in embodiment b above, is reduced to obtain corresponding compounds of the formula VIIb. Said reduction reaction is carried out in a manner known to the person skilled in the art, for example as described in J. Org. Chem. 1962, 27, 4426 or as described in the following examples. More specifically, the reduction can be carried out, for example, by contacting compounds of the formula VIIIb with a hydrogen-producing mixture such as, preferably, metallic zinc in a mildly acidic medium such as acetic acid in a lower alcohol such as methanol or ethanol at room temperature or at elevated temperature or, preferably, at the boiling temperature of the solvent mixture. Alternatively, the reduction can be carried out by selective reduction of the nitro group in a manner known to the person skilled in the art, for example by hydrogen transfer reaction in the presence of a metal catalyst, for example palladium or preferably Raney nickel, in a suitable solvent, preferably a lower alcohol, using, for example ammonium formiate or preferably hydrazine hydrate as hydrogen donor.

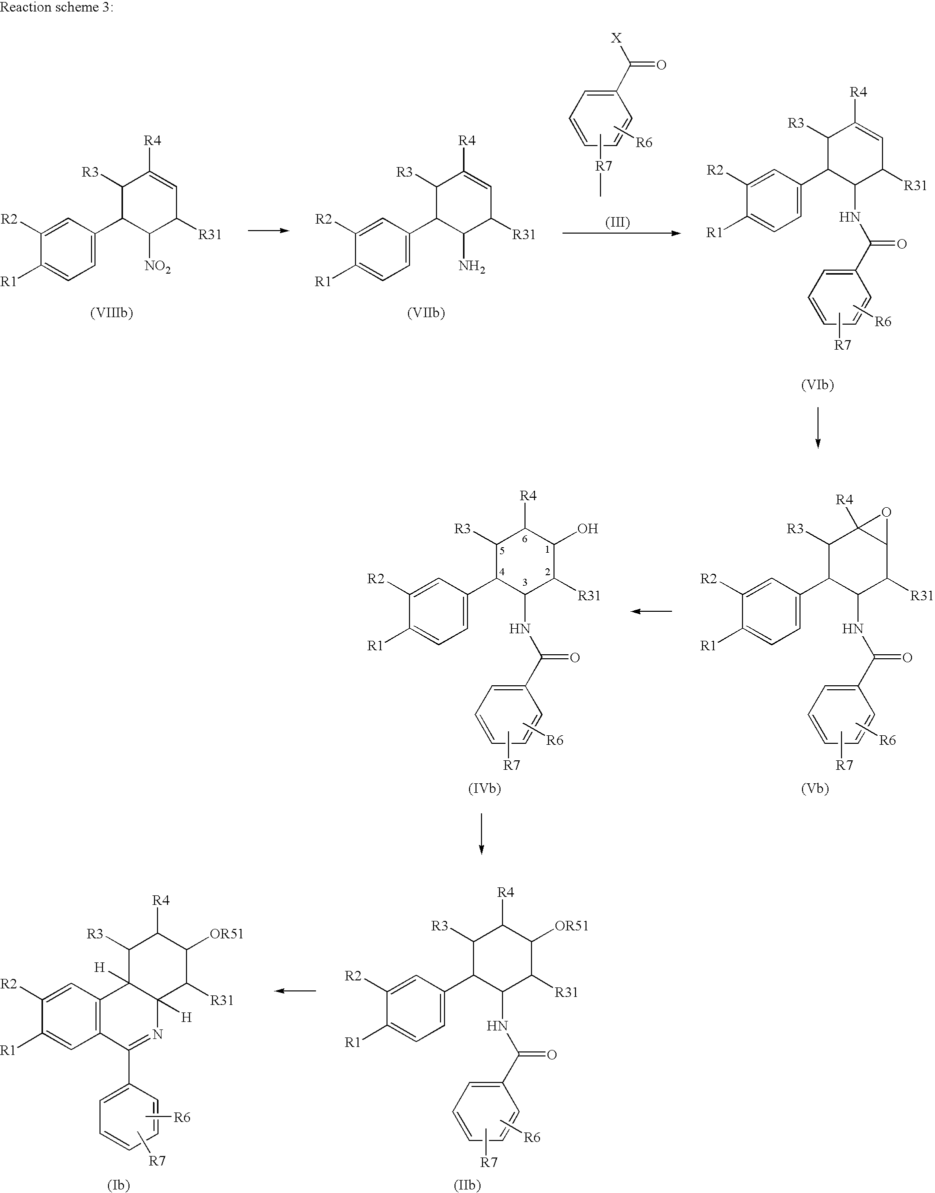

Compounds of the formula VIIb obtained can be reacted, for example, as described by way of example in the following examples with compounds of the formula III, in which R6 and R7 have the meanings given above and X represents a suitable leaving group, preferably a chlorine atom, to give corresponding compounds of the formula VIb.

Alternatively, compounds of the formula VIb, in which R1, R2, R3, R31, R4, R6 and R7 have the meanings given above in embodiment b, can also be prepared, for example, from corresponding compounds of the formula VIIb and corresponding compounds of the formula III, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1-yl)-N,N,N′,N′-tetramthyl-uroniumhexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

In the next step compounds of the formula VIb are converted into corresponding compounds of the formula Vb by epoxidation reaction, which can be carried out as described in the following examples or in a manner known to one of ordinary skill in the art employing, for example, suitable epoxidation methods or suitable epoxidation reagents such as, for example, peracids (e.g. m-chloroperbenzoic acid) or organic or inorganic peroxides (e. g. dimethyldioxirane, hydrogene peroxide or persulfates).

Compounds of the formula Vb obtained can be reduced by art-known methods to corresponding compounds of the formula IVb. More specifically, said reduction reaction can be performed employing, for example, as described by way of example in the following examples sodium borohydride as reductant. Alternatively, said reduction reaction can be also carried out using, for example, lithium aluminium hydride or a reductive mixture comprising noble metals, such as platinium dioxide or palladium, and a suitable hydrogen donor. With the aid of each of those said reduction methods, compounds of the formula Vb can be converted largely regio- and diastereoselectively into compounds of the formula IVb, wherein the hydroxyl radical in position 1 and the amido radical in position 3 are located at the same side of the plane defined by the cyclohexane ring.

It is moreover known to one of ordinary skill of the art, that the absolute configuration of a chiral carbon atom, preferably, to which a hydroxyl group and a hydrogen atom are bonded, can be inverted. Thus the configuration of the carbon atom in position 1 of compounds of the formula IVb can be optionally inverted. Said inversion of configuration of position 1 of compounds of the formula IVb can be achieved in a manner familiar to the person skilled in the art, for example by derivatization of position 1 with a suitable leaving group and subsequent replacement of said leaving group by a suitable nucleophile in a nucleophilic substitution reaction according to SN2 mechanism. Alternatively, said inversion of configuration of position 1 of compounds of the formula IVb can be also obtained, for example, as described by way of example in the following examples according to subsequently specified two step procedure shown in reaction scheme 4 below. In more detail, in the first step of said procedure shown in reaction scheme 4, exemplary compounds of the formula IVb*, in which R1, R2, R6 and R7 have the meanings indicated above in embodiment b, and R3, R31 and R4 are hydrogen and position 1 has the R configuration, are converted by oxidation reaction into corresponding compounds of the formula IXb. Said oxidation is likewise carried out under conditions customary per se using, for example, chloranil, atmospheric oxygen, manganese dioxide or, preferably, chromium oxides as an oxidant. Then in the second step, compounds of the formula IXb obtained are converted by art-known reduction reaction of the keto group, preferably with metal hydride compounds or, more specifically, metal borohydrides, such as, for example, sodium borohydride, into corresponding compounds of formula IVb**, in which position 1 has now S configuration and thus the configuration of the carbon atom in position 1 is now inverted regarding to said compounds of the formula IVb*.

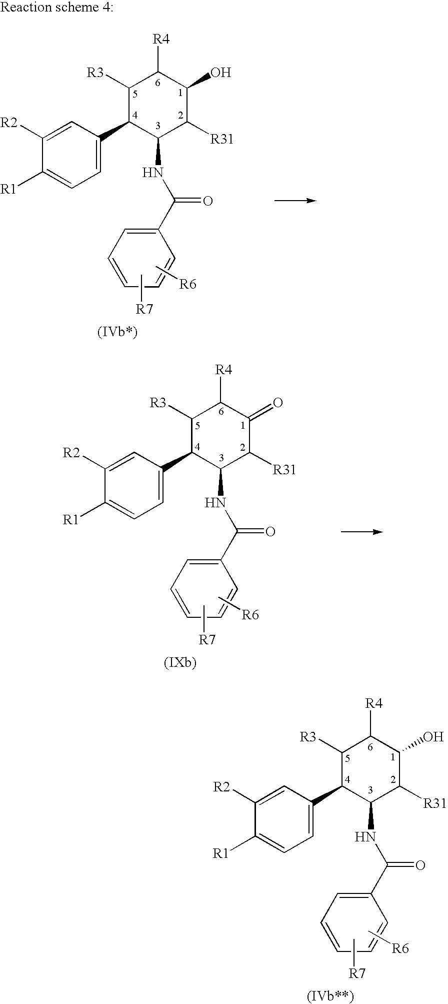

In the next reaction step of the synthesis route shown in reaction scheme 3 shown above, compounds of the formula IVb are converted into corresponding compounds of the formula IIb by introduction of the group R51 whereby R51 is other than hydrogen. The introduction reaction is carried out in a manner habitual per se (e.g. via alkylation or acylation reaction) or as described by way of example in the following examples.

The cyclization reaction leading to compounds of the formula Ib, in which R1, R2, R3, R31, R4, R51, R6 and R7 have the meanings given above in embodiment b whereby R51 is other than hydrogen, can be carried out, for example, as described by way of example in the following examples or analogously or similarly thereto, or as mentioned above for compounds according to embodiment a.

Compounds of the formula VIIIb, in which R1, R2, R3, R31 and R4 have the meanings mentioned above in embodiment b, are either known or can be obtained, for example as shown in reaction scheme 5, by the reaction of compounds of the formula IXa, in which R1 and R2 have the abovementioned meanings, with compounds of the formula Xb, in which R3, R31 and R4 have the meanings indicated above in embodiment b.

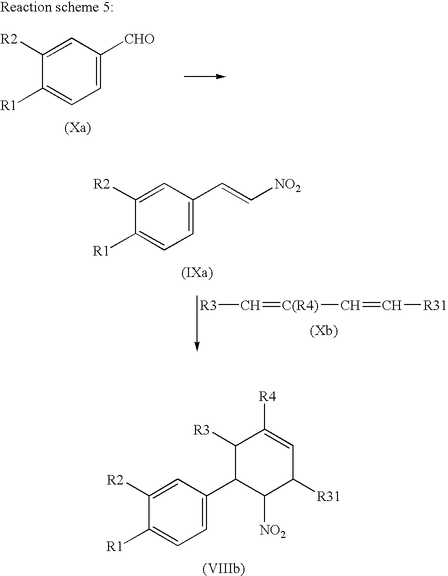

The cycloaddition is in this case carried out in a manner known to the person skilled in the art according to Diels-Alder, e.g. as described in J. Amer. Chem. Soc. 1957, 79, 6559 or In J. Org. Chem. 1952, 17, 581 or as described in the following examples.

Compounds of the formula VIIIb, in which the phenyl ring and the nitro group are trans to one another, can be converted such as known to the person skilled in the art into the corresponding cis compounds, e.g. as described in J. Amer. Chem. Soc. 1957, 79, 6559 or as described in the following examples.

The compounds of the formula Xb are either known or can be prepared in a known manner.

In an alternative, compounds of the formula IIb, in which R1, R2, R3, R31, R4, R51, R6 and R7 have the meanings given above in embodiment b whereby R51 is other than hydrogen (particularly compounds of formula IIb, in which R1, R2, R51, R6 and R7 have the meanings given above in embodiment b whereby R51 is other than hydrogen, and R3, R31 and R4 are all hydrogen) can also be obtained as shown in reaction scheme 6 and as described by way of example in the following examples.

In the first reaction step of the route outlined in reaction scheme 6, the amino group of compounds of the formula VIIb is protected with an art-known protective group PG1, such as e.g. the tert-butoxycarbonyl group. The proteced compounds are subjected to hydroboration reaction to obtain over two steps compounds of formula XIb. Said hydroboration reaction is carried out as described in the following examples using an appropriate (hydro)borating agent, such as e.g. 9-BBN, isopinocampheylborane or the like, or, particularly, borane-tetrahydrofuran (H₃B-THF), advantageously at ambient temperature. The compounds obtained are then converted into compounds of the formula XIb by introduction of the group R51 whereby R51 is other than hydrogen in a manner analogously as described above.

In the next reaction step of the synthesis route shown in reaction scheme 6, compounds of formula XIb are converted into corresponding compounds of the formula IIb by deprotection of the protective group PG1 and amidification with compounds of the formula III. Said reactions are carried out in a manner habitual per se or as described in the specification of this invention or in the following examples.

If necessary, the product obtained via said hydroboration reaction or, suitably, the R51-substituted derivative thereof is purified from resulting stereo- and/or regioisomeric side products by methods known to the person skilled in the art, such as e.g. by chromatographic separation techniques.

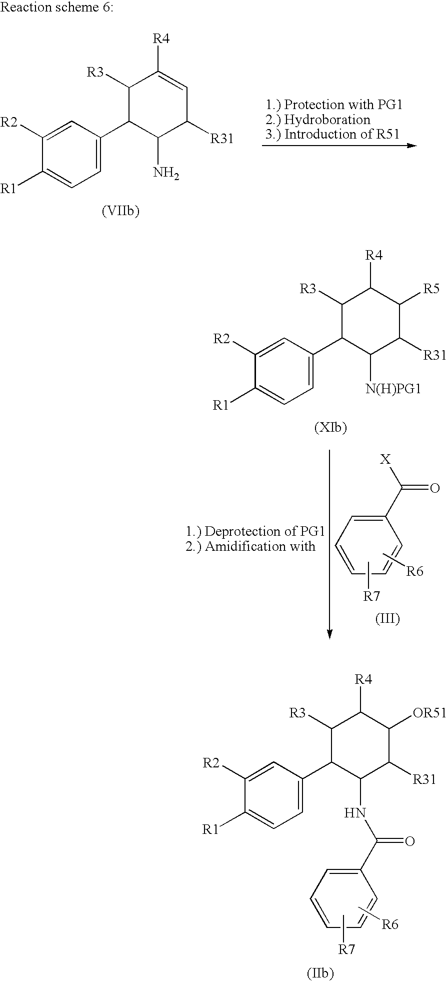

Alternatively, compounds of formula I according to aspect 2 (compounds of formula I′ as shown below), in which A is a bond, can be obtained as outlined in reaction scheme 1′ and as described as follows starting from the compounds of formula III′, in which R1, R2, R3, R31, R4, R5 and R6 have the meanings mentioned above whereby R4 and R5 are other than hydroxyl, by reduction of the nitro group of compounds of formula III′ to give corresponding compounds of formula II′. Said reduction can be carried out in a manner known to the person skilled in the art or as described exemplarily in the following examples, for example, by hydrogenation in the presence of a suitable metal catalyst, or, particularly, using a suitable reducing agent such as e.g. tin dichloride.

Compounds of formula II′ and their preparation are also known from WO2004/019944 or WO2004/019945, the disclosure of which is incorporated herein.

In the next step compounds of formula II′ are reacted with sulphonic acid derivatives of the formula R11-S(O)₂—X, in which X is a suitable leaving group, for example a chlorine atom, to give the corresponding sulphonylamino compounds. Optionally, said sulphonylamino compounds can be N-alkylated using a suitable base and a suitable alkylating agent R10-Y, in which Y is a suitable leaving group, in an one-pot or a sequential reaction (first deprotonation reaction than addition of R10-Y).

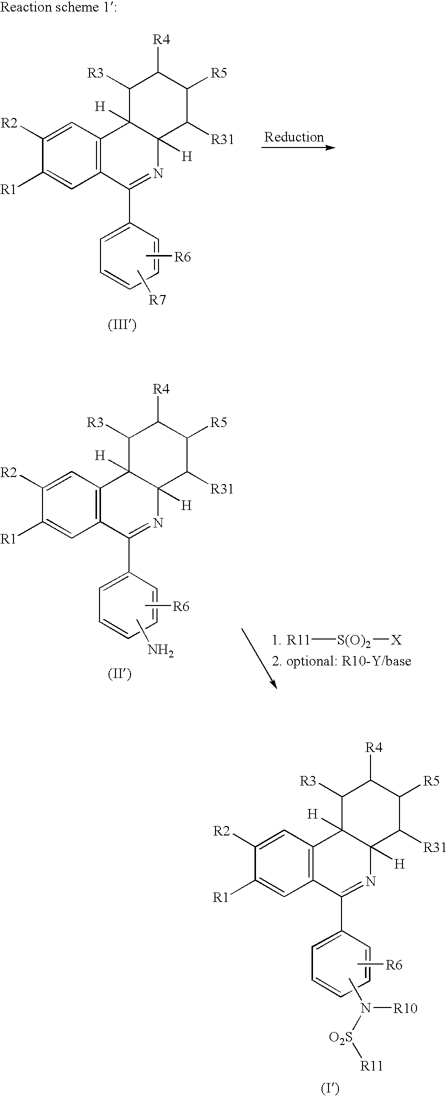

Compounds of formula III′ can be obtained analogously as shown in the reaction schemes 1 to 5 and specified above thereto, or in an analogous or similar manner as described in the following examples.

Optionally, compounds of the formula I can be also converted into further compounds of the formula I by methods known to one of ordinary skill in the art. More specifically, for example, from compounds of the formula I in which
a) R41 or R51 is hydrogen, the corresponding ester compounds can be obtained by esterification reactions;
b) R41 or R51 is hydrogen, the corresponding ether compounds can be obtained by etherification reactions;
c) R41 or R51 is an acyl group, such as e.g. acetyl, the corresponding hydroxyl compounds can be obtained by deesterification (e.g. saponification) reactions;
d) R10 is hydrogen, the corresponding N-ether compounds can be obtained by etherification reactions,
e.) R7 is —SR12, the corresponding sulfoxides or sulfones can be obtained by mono- or bis-oxidation of the sulphur atom.

The methods mentioned under a), b), c), d.) and e) are expediently carried out analogously to the methods known to the person skilled in the art or as described by way of example in the following examples.

Optionally, compounds of the formula I can be converted into their salts, or, optionally, salts of the compounds of the formula I can be converted into the free compounds.

In addition, the compounds of the formula I can be converted, optionally, into their N-oxides, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds of the formula I, whose preparation is not explicitly described, can be prepared in an analogous or similar manner or in a manner familiar per se to the person skilled in the art using customary process techniques.

Any or all of the compounds which are mentioned in the following examples as final compounds as well as their salts, N-oxides and salts of the N-oxides are a preferred subject of the present invention.

In the examples, m.p. stands for melting point, h for hour(s), min for minutes, R_{f} for rentention factor in thin layer chromatography, s.p. for sintering point, EF for empirical formula, MW for molecular weight, MS for mass spectrum, M for molecular ion, fnd. for found, calc. for calculated, other abbreviations have their meanings customary per se to the skilled person.

According to common practice in stereochemistry, the symbols RS and SR are used to denote the specific configuration of each of the chiral centers of a racemate. In more detail, for example, the term “(2RS,4aRS,10bRS)” stands for a racemate (racemic mixture) comprising the one enantiomer having the configuration (2R,4aR,10bR) and the other enantiomer having the configuration (2S,4aS,10bS).

### EXAMPLES

Final Compounds

Starting from the appropriate ester compounds, which are mentioned or described explicitly below (compounds 13 to 22), or which can be prepared in a manner known to the person skilled in the art or analogously or similarly to the examples described herein, the compounds 1 to 10 are obtained according to the procedure as in Example 11.

### 1. N-[4-((2RS,4aRS,10bRS)-2-Hydroxy-8,9-dimethoxy-1,2,3,4,4a,10b- -hexahydro-phenanthridin-6yl)-phenyl]-4,N-dimethyl-benzenesulfonamide

EF: C₂₉H₃₂N₂O₅S MW: 520,65 MS: 521,2(MH⁺)

### 2. 4-Fluoro-N-[4-(2RS,4aRS,10bRS)-2-hydroxy-8,9-dimethoxy-1,2,3,4,4a,10b-hexahydrophenanthridin-6-yl)-phenyl]-benzenesulfonamide

EF: C₂₇H₂₇F N₂O₅S MW: 510,59 MS: 511,2 (MH⁺)

### 3. N[4-((2RS,4aRS,10bRS)-2-Hydroxy-8,9-dimethoxy-1,2,3,4,4a,10b-hexahydro-phenanthridin-6-yl)-2-methyl-phenyl]-4-methyl-benzenesulfonamide

EF: C₂₉H₃₂N₂O₅S MW: 520,65 MS: 521,3 (MH⁺)

### 4. N[4-(2RS,4aRS,10bRS)-2-Hydroxy-8,9-dimethoxy-1,2,3,4,4a,10b-hexahydro-phenanthridin-6-yl)-benzyl]-4-methyl-benzenesulfonamide

EF: C₂₉H₃₂N₂O₅S MW: 520,65 MS: 521,3 (MH⁺)

### 5. N{4-[(2RS,4aRS,10bRS-8-(1,1-Difluoro-methoxy)-2-hydroxy-9-methoxy-1,2,3,4,4a,10b-hexahydro-phenanthridin-6-yl]-phenyl}-methanesulfonamide

### 6. N-{4-[(2RS,4aRS,10bRS)-8-(1,1-Difluoro-methoxy)-2-hydroxy-9-methoxy-1,2,3,4,4a,10b-hexahydro-phenanthridin-6-yl]-phenyl}-4-methyl-benzenesulfonamide

EF: C₂₈H₂₈F₂N₂O₅S MW: 542,61 MS: 543,3 (MH⁺)

### 7. N-{4-[(2RS,4aRS,10bRS)-8-(1,1-Difluoro-methoxy)-2-hydroxy-9-methoxy-1,2,3,4,4a,10b-hexahydro-phenanthridin-6-yl]-phenyl}-4-fluoro-benzenesulfonamide

EF: C₂₇H₂₅F₃N₂O₅S MW: 546,57 MS: 547,2 (MH⁺)

### 8. N-{4-[(2RS,4aRS,10bRS)-8-(1,1-Difluoro-methoxy)-2-hydroxy-9-methoxy-1,2,3,4,4a,10b-hexahydro-phenanthridin-6yl]-benzyl}-4-methyl-benzenesulfonamide

EF: C₂₉H₃₀F₂N₂O₅S MW: 556,63 MS: 557,4 (MH⁺)

### 9. N-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-4-methyl-benzenesulfonamide

EF: C₂₈H₂₈F₂N₂O₅S MW: 542,61 MS: 543,3 (MH⁺)

### 10. N-{4-[(2RS,4aRS,10bRS)-9-(1,1-Difluoro-methoxy)-2-hydroxy-8-methoxy-1,2,3,4,4a,10b-hexahydro-phenanthridin-6-yl]-benzyl}-4-methyl-benzenesulfonamide

EF: C₂₉H₃₀F₂N₂O₅S MW: 556,63 MS: 557,4 (MH⁺)

### 11. N-[4-((2RS,4aRS,10bRS)-2-Hydroxy-8,9-dimethoxy-1,2,3,4,4a,10b-hexahydro-phenanthridin-6-yl)-phenyl]-4-methyl-benzenesulfonamide

440 mg of acetic acid (2RS,4aRS,10bRS)-8,9-dimethoxy-6-[4-toluene-4-sulfonylamino)-phenyl]-1,2,3,4,4a,10b-hexahydro-phenanthridin-2-yl ester (compound 12) and 136 mg of cesium carbonate are dissolved in 2 ml of dichloromethane and 14 ml of methanol. The solution is stirred for 16 h. Again 136 mg of cesium carbonate are added and the mixture stirred for another 24 h. Then the solvent is removed and the residue purified by flash chromatography to give 204 mg of the title compound.

Starting from the appropriate starting compounds, which are mentioned or described explicitly below, or which can be prepared in a manner known to the person skilled in the art or analogously or similarly to the examples described herein, further relevant, non-explicitly described compounds which are analogues of compound 11 are obtained according to the procedure as in Example 11.

### 12. Acetic acid (2RS,4aRS,10bRS)-8,9-dimethoxy-6-[4-toluene-4-sulfonylamino)-phenyl]-1,2,3,4,4a,10b-hexahydro-phenanthridin-2-yl ester

1.417 g of phosphorus pentachloride are suspended in 50 ml of dichloromethane. 963 mg of A1 dissolved in 30 ml of dichloromethane are added as well as 10 ml of isoprpopylacetate. The reaction mixture is stirred at 50° C. for 6 h then at room temperature for 16 h. A mixture of 25 ml of dichloromethane and 25 ml of triethylamine is added, than cautiously 25 ml of water with vigorous strirring. After rextraction with dichloromethane the organic layer is dried over magnesium sulfate and the crude product purified by flash chromatography to give 670 mg of the title compound.

EF: C₃₀H₃₂N₂O₆S MW: 548,66 MS: 549,3 (MH⁺)

Starting from the appropriate starting compounds, which can be prepared in a manner known to the person skilled in the art or analogously or similarly to the examples described herein below, the following compounds are obtained analogously or similarly to the procedure described in Example 12. If necessary, the cyclization reaction can be carried out in the presence of a catalytic amount of a Lewis acid such e.g. tin tetrachloride.

### 13. Acetic acid (2RS,4aRS,10bRS)-8,9-dimethoxy-6-{4-methyl-(toluene-4-sulfonyl)-amino]-phenyl}-1,2,3,4,4a,10b-hexahydro-phenanthridin-2-yl ester

EF: C₃₁H₃₄N₂O₆S MW: 562,69 MS: 563,3 (MH⁺)

### 14. Acetic acid (2RS,4aRS,10bRS)[-6-[4-(4-fluoro-benzenesulfonylamino)-phenyl]-8,9-dimethoxy-1,2,3,4,4a,10b-hexahydro-phenanthridin-2-yl ester

EF: C₂₉H₂₉F N₂O₆S MW: 552,63 MS: 553,3 (MH⁺)

### 15. Acetic acid (2RS,4aRS,10bRS)-8,9-dimethoxy-6-[3-methyl-4-(toluene-4-sulfonylamino)-phenyl]-1,2,3,4,4a,10b-hexahydro-phenanthridin-2-yl ester

EF: C₃₁H₃₄N₂O₆S MW: 562,69 MS: 563,4 (MH⁺)

### 16. Acetic acid (2RS,4aRS,10bRS)-8,9-dimethoxy-6-{4-[(toluene-4-sulfonylamino)-methyl]-phenyl]-1,2,3,4,4a,10b-hexahydro-phenanthridin-2-yl ester

EF: C₃₁H₃₄N₂O₆S MW: 562,69 MS: 563,4 (MH⁺)

### 17. Acetic acid (2RS,4aRS,10bRS)-8-(1,1-difluoro-methoxy)6-(4methanesulfonylamino-phenyl)-9-methoxy-1,2,3,4,4a,10b-hexahydro-phenanthridin-2-yl ester

EF: C₂₄H₃₀F₂N₂O₆S MW: 508,55 MS: 509,3 (MH⁺)

### 18. Acetic acid (2RS,4aRS,10bRS)-8-(1,1-difluoro-methoxy)-9-methoxy-6-[4-(toluene-4-sulfonylamino)-phenyl]-1,2,3,4,4a,10b-hexahydro-phenanthridin-2-yl ester

EF: C₃₀H₃₀F₂N₂O₆S MW: 584,64 MS: 585,3 (MH⁺)

### 19. Acetic acid (2RS,4aRS,10bRS)-8-(1,1-difluoro-methoxy)-6-[4-(4fluoro-benzenesulfonylamino)-phenyl]-9-methoxy-1,2,3,4,4a,10b-hexahydro-phenanthridin-2-yl ester

EF: C₂₉H₂₇F₃N₂O₆S MW: 588,61 MS: 589,3 (MH⁺)

### 20. Acetic acid (2RS,4aRS,10bRS)-8(1,1-difluoro-methoxy)-9-methoxy-6-{4-[(toluene-4-sulfonylamino)-methyl]-phenyl}-1,2,3,4,4a,10b-hexahydro-phenanthridin-2-yl ester

EF: C₃₁H₃₂F₂N₂O₆S MW: 598,67 MS:599,3 (MH⁺)

### 21. Acetic acid (2RS,4aRS,10bRS)-9-(1,1-difluoro-methoxy)-8-methoxy-6-[4-toluene-4-sulfonylamino)-phenyl]-1,2,3,4,4a,10b-hexahydro-phenanthridin-2-yl ester

EF: C₃₀H₃₀F₂N₂O₆S MW: 584,64 MS: 585,3 (MH⁺)

### 22. Acetic acid (2RS,4aRS,10bRS)-9-(1,1-difluoro-methoxy)-8-methoxy-6-{4-[(toluene-4-sulfonylamino)-methyl]-phenyl]-1,2,3,4,4a,10b-hexahydro-phenanthridin-2-yl ester

EF: C₃₁H₃₂F₂N₂O₆S MW: 598,67 MS: 599,3 (MH⁺)

Starting from the appropriate ester compounds, which are mentioned or described explicitly below (compounds 43 to 62), or which can be prepared in a manner known to the person skilled in the art or analogously or similarly to the examples described herein, the compounds 23 to 42 are obtained according to the procedure as in Example 11.

### 23. 4-((2RS,4aRS,10bRS)-2-Hydroxy-8,9-dimethoxy-1,2,3,4,4a,10b-hexahydro-phenanthridin-6-yl)-N,N-dipropyl-benzenesulfonamide

C₂₇H₃₆N₂O₅S MW: 500,66 MS: 501,4 (MH⁺)

### 24. 4-((2RS,4aRS,10bRS)-9-Ethoxy-2-hydroxy-8-methoxy-1,2,3,4,4a,10b-hexahydro-phenanthridin-6-yl)-N,N-dipropyl-benzenesulfonamide

C₂₈H₃₀N₂O₅S MW: 514,69 MS: 515,4 (MH⁺)

### 25. 4-((2RS,4aRS,10bRS)-9-Ethoxy-2-hydroxy-8-methoxy-1,2,3,4,4a,10b-hexahydro-phenanthridin-6-yl-N-(2-methoxy-ethyl)-N-methyl-benzenesulfonamide

C₂₆H₃₄N₂O₆S MW: 502,63 MS: 503,3 (MH⁺)

### 26. (2RS,4aRS,10bRS)-8-(1,1-Difluoro-methoxy)-9-methoxy-6-[4-(pyrrolidine-1-sulfonyl)-phenyl]-1,2,3,4,4a,10b-hexahydro-phenanthridin-2-ol

C₂₅H₂₈F₂N₂O₅S MW: 506,57 MS: 507,3 (MH⁺)

### 27. (2RS,4aRS,10bRS)-8-(1,1-Difluoro-methoxy)-9-methoxy-6-[4-(piperidine-1-sulfonyl)-phenyl]-1,2,3,4,4a,10b-hexahydro-phenanthridin-2-ol

C₂₆H₃₀F₂N₂O₅S MW: 520,6 MS: 521,4 (MH⁺)

### 28. (2RS,4aRS,10bRS)-8-(1,1-Difluoro-methoxy)-9-methoxy-6-[3-(pyrrolidine-1-sulfonyl)-phenyl]-1,2,3,4,4a,10b-hexahydro-phenanthridin-2-ol

C₂₅H₂₈F₂N₂O₅S MW: 506,57 MS: 507,4 (MH⁺)

### 29. 4-[(2RS,4aRS,10bRS)-8-(1,1-Difluoro-methoxy)-2-hydroxy-9-methoxy-1,2,3,4,4a,10b-hexahydro-phenanthridin-6-yl]-N,N-dipropyl-benzenesulfonamide

C₂₇H₃₄F₂N₂O₅S MW: 536,64 MS: 537,4 (MH⁺)

### 30. (2RS,4aRS,10bRS)-8-(1,1-Difluoro-methoxy)-9-methoxy-6-[4-(morpholine-4-sulfonyl)-phenyl]-1,2,3,4,4a,10b-hexahydro-phenanthridin-2-ol

C₂₅H₂₈F₂N₂O₆S MW: 522,57 MS: 523,3 (MH⁺)

### 31. (2RS,4aRS,10bRS)-8-(1,1-Difluoro-methoxy)-9-methoxy-6-[3-(morpholine-4-sulfonyl)-phenyl]-1,2,3,4,4a,10b-hexahydro-phenanthridin-2-ol

C₂₅H₂₈F₂N₂O₆S MW: 522,57 MS: 523,4 (MH⁺)

### 32. (2RS,4aRS,10bRS)-8-(1,1-Difluoro-methoxy)-9-methoxy-6-[3-(piperidine-1-sulfonyl)-phenyl]-1,2,3,4,4a,10b-hexahydro-phenanthridin-2-ol

C₂₈H₃₀F₂N₂O₅S MW: 520,6 MS: 521,3 (MH⁺)

### 33. 3-[(2RS,4aRS,10bRS)-8-(1,1-Difluoro-methoxy)-2-hydroxy-9-methoxy-1,2,3,4,4a,10b-hexahydro-phenanthridin-6-yl]-N,N-dimethyl-benzenesulfonamide

C₂₉H₂₆F₂N₂O₆S MW: 480,53 MS: 481,3 (MH⁺)

### 34. N-Cyclopropyl-3-[(2RS,4aRS,10bRS-8-(1,1-difluoro-methoxy)-2-hydroxy-9-methoxy-1,2,3,4,4a,10b-hexahydro-phenanthridin-6-yl]-benzenesulfonamide

C₂₄H₂₆F₂N₂O₅S MW: 492,55 MS: 493,3 (MH⁺)

### 35. 3-[(2RS,4aRS,10bRS)-8-(1,1-Difluoro-methoxy)-2-hydroxy-9-methoxy-1,2,3,4,4a,10b-hexahydro-phenanthridin-6-yl]-N,N-bis-(2-methoxy-ethyl)-benzenesulfonamide

C₂₇H₃₄F₂N₂O₇S MW: 568,64 MS: 569,3 (MH⁺)

### 36. (2RS,4aRS,10bRS)-8-(1,1-Difluoro-methoxy)-9-methoxy-6-[3-(4-methyl-piperazine-1-sulfonyl)-phenyl]-2,3,4,4a,10b-hexahydro-phenanthridin-2-ol

C₂₈H₃₁F₂N₃₅S MW: 535,61 MS: 536,3 (MH⁺)

### 37. (2RS,4aRS,10bRS)-9-(2,2-Difluoro-ethoxy)-8-methoxy-6-[3-4-methyl-piperazine-1-sulfonyl)-phenyl]-1,2,3,4,4a,10b-hexahydro-phenanthridin-2-ol

C27 H33 F2 N3 O5 S MW: 549,64 MS: 550,3 (MH⁺)

### 38. (2RS,4aRS,10bRS)-9-(2,2-Difluoro-ethoxy)-8-methoxy-6-[4-(pyrrolidine-1-sulfonyl)-phenyl]-1,2,3,4,4a,10b-hexahydro-phenanthridin-2-ol

C26 H30 F2 N2 O5 S MW: 520,6 MS: 521,3 (MH⁺)

### 39. (2RS,4aRS,10bRS)-6-(3-Methanesulfonyl-phenyl)-8,9-dimethoxy-1,2,3,4,4a,10b-hexahydro-phenanthridin-2-ol

C₂₂H₂₅N O₅S MW: 415,51 MS: 416,3 (MH⁺)

### 40. (2RS,4aRS,10bRS)-9-Ethoxy-8-methoxy-6-(4-methylsulfanyl-phenyl)-1,2,3,4,4a,10b-hexahydro-phenanthridin-2-ol

C₂₃H₂₇N O₃S MW: 397.54 MS: 398,2 (MH⁺)

### 41. (2R,4aR,10bR)-9-Ethoxy-8-methoxy-6-4-methylsulfanyl-phenyl)-1,2,3,4,4a,10b-hexahydro-phenanthridin-2-ol

C₂₃H₂₇N O₃S MW: 397,54 MS: 398,3 (MH⁺)

### 42. (2RS,4aRS,10bRS)-9-(2,2-Difluoro-ethoxy)-8-methoxy-6-(4-methylsulfanyl-phenyl)-1,2,3,4,4a,10b-hexahydro-phenanthridin-2-ol

C23 H25 F2 N O3 S MW: 433,52 MS: 434,2 (MH⁺)

Starting from the appropriate starting compounds which can be obtained in analogy to the below-described ones, the following and further relevant ester compounds can be obtained according to the cyclization reactions described in Example 12 or analogously or similarly thereto. If necessary, the cyclization reaction can be carried out in the presence of a catalytic amount of a Lewis acid such e.g. tin tetrachloride.

### 43. Acetic acid (2RS,4aRS,10bRS)-6-(4dipropylsulfamoyl-phenyl)-8,9-dimethoxy-1,2,3,4,4a,10b-hexahydro-phenanthridin-2-yl ester

C₂₉H₃₈N₂O₆S MW: 542,7 MS: 543,4 (MH⁺)

### 44. Acetic acid (2RS,4aRS,10bRS)-6-(4-dipropylsulfamoyl-phenyl)-9-ethoxy-8-methoxy-1,2,3,4,4a,10b-hexahydro-phenanthridin-2-yl ester

C₃₀H₄₀N₂O₆S MW: 556,73 MS: 557,4 (MH⁺)

### 45. Acetic acid (2RS,4aRS,10bRS)-9-ethoxy-8-methoxy-6-{4-[(2-methoxy-ethyl)-methyl-sulfamoyl]-phenyl}-1,2,3,4,4a,10b-hexahydro-phenanthridin-2-yl ester

C₂₈H₃₈N₂O₇S MW: 544,67 MS: 545,3 (MH⁺)

### 46. Acetic acid (2RS,4aRS,10bRS)-8-(1,1-difluoro-methoxy)-9-methoxy-6-[4-(pyrrolidine-1-sulfonyl)-phenyl]-1,2,3,4,4a,10b-hexahydro-phenanthridin-2-yl ester

C₂₇H₃₀F₂N₂O₆S MW: 548,61 MS: 549,3 (MH⁺)

### 47. Acetic acid (2RS,4aRS,10bRS)-8-(1,1-difluoro-methoxy)-9-methoxy-6-[4-(piperidine-1-sulfonyl)-phenyl]-1,2,3,4,4a,10b-hexahydro-phenanthridin-2-yl ester

### 48. Acetic acid (2RS,4aRS,10bRS)-8-(1,1-difluoro-methoxy)-9-methoxy-6-[3-(pyrrolidine-1-sulfonyl)-phenyl]-1,2,3,4,4a,10b-hexahydro-phenanthridin-2-yl ester

C₂₇H₃₀F₂N₂O₆S MW: 548,61 MS: 549,3 (MH⁺)

### 49. Acetic acid (2RS,4aRS,10bRS)-8-(difluoro-methoxy)-6-(4-dipropylsulfamoyl-phenyl)9-methoxy-1,2,3,4,4a,10b-hexahydro-phenanthridin-2-yl ester

C₂₉H₃₆F₂N₂O₅S MW: 578,68 MS: 579,3 (MH⁺)

### 50. Acetic acid (2RS,4aRS,10bRS)-8-(1,1-difluoro-methoxy)-9-methoxy-6-[4-(morpholine-4-sulfonyl)-phenyl]-2,3,4,4a,10b-hexahydro-phenanthridin-2-yl ester

### 51. Acetic acid (2RS,4aRS,10bRS)-8-(1,1-difluoro-methoxy)-9-methoxy-6-[3-(morpholine-4-sulfonyl)-phenyl]-1,2,3,4,4a,10b-hexahydro-phenanthridin-2-yl ester

C₂₇H₃₀F₂N₂O₇S MW: 564,61 MS: 565,3 (MH⁺)

### 52. Acetic acid (2RS,4aRS,10bRS)-8-(1,1-difluoro-methoxy)-9-methoxy-6-[3-(piperidine-1-sulfonyl)-phenyl]1,2,3,4,4a,10b-hexahydro-phenanthridin-2-yl ester

### 53. Acetic acid (2RS,4aRS,10bRS)-8-(difluoro-methoxy)-6-(3-dimethylsulfamoyl-phenyl)-9-methoxy-1,2,3,4,4a,10b-hexahydro-phenanthridin-2-yl ester

C₂₅H₂₈F₂N₂O₆S MW: 522,57 MS: 523,3 (MH⁺)

### 54. Acetic acid (2RS,4aRS,10bRS)-6-(3-cyclopropylsulfamoyl-phenyl)-8-(1,1-difluoro-methoxy)-9-methoxy-1,2,3,4,4a,10b-hexahydro-phenanthridin-2-yl ester

C₂₆H₂₈F₂N₂O₆S MW: 534,58 MS: 535,5 (MH⁺)

### 55. Acetic acid (2RS,4aRS,10bRS)-6-{3-[bis-2-methoxy-ethyl)-sulfamoyl]-phenyl}-8-(difluoro-methoxy)-9-methoxy-1,2,3,4,4a,10b-hexahydro-phenanthridin-2-yl ester

C₂₉H₃₆F₂N₂O₈S MW: 610,68 MS: 611,3 (MH⁺)

### 56. Acetic acid (2RS,4aRS,10bRS)-8-(1,1-difluoro-methoxy)-9-methoxy-6-[3-(4-methyl-piperazine-1-sulfonyl)-phenyl]1,2,3,4,4a,10b-hexahydro-phenanthridin-2-yl ester

C₂₈H₃₃F₂N₃O₆S MW: 577,65 MS: 578,3 (MH⁺)

### 57. Acetic acid (2RS,4aRS,10bRS)-9-2,2-difluoro-ethoxy)-8-methoxy-6-[3-4methyl-piperazine-1-sulfonyl)-phenyl]-1,2,3,4,4a,10b-hexahydro-phenanthridin-2-yl ester

### 58. Acetic acid (2RS,4aRS,10bRS)-9-(2,2-difluoro-ethoxy)-8-methoxy-6-[4-(pyrrolidine-1-sulfonyl)-phenyl]-1,2,3,4,4a,10b-hexahydro-phenanthridin-2-yl ester

### 59. Acetic acid (2RS,4aRS,10bRS)-6-(3-methanesulfonyl-phenyl)-8,9-dimethoxy-1,2,3,4,4a,10b-hexahydro-phenanthridin-2-yl ester

C₂₄H₂₇N O₈S MW: 457,55 MS: 458,2 (MH⁺)

### 60. Acetic acid (2RS,4aRS,10bRS)-9-ethoxy-8-methoxy-6-(4methylsulfanyl-phenyl)-1,2,3,4,4a,10b-hexahydro-phenanthridin-2-yl ester

C₂₅H₂₉N O₄S MW: 439,58 MS: 440,2 (MH⁺)

### 61. Acetic acid (2R,4aR,10bR)-9-ethoxy-8-methoxy-6-(4-methylsulfanyl-phenyl1,2,3,4,4a,10b-hexahydro-phenanthridin-2-yl ester

C25 H29 N O4 S MW: 439,58 MS: 440,3 (MH⁺)

### 62. Acetic acid (2RS,4aRS,10bRS)-9-2,2-difluoro-ethoxy)-8-methoxy-6-(4-methylsulfanyl-phenyl)-1,2,3,4,4a,10b-hexahydro-phenanthridin-2-yl ester

### 63. (2R,4aR,10bR)-9-Ethoxy-8-methoxy-6-(3-methylsulfanyl-phenyl)-1,2,3,4,4a,10b-hexahydro-phenanthridin-2-ol

The title compound can be obtained via an analogous or similar synthesisis route as described for Example 41.

The following compounds may be obtained in a manner analogously as described herein via art-known mono-S-oxidation at an appropriate stage of the synthesis route.

### 64. (2R,4aR,10bR)-9-Ethoxy-8-methoxy-6-(4methylsulfinyl-phenyl)-1,2,3,4,4a,10b-hexahydro-phenanthridin-2-ol

### 65. (2R,4aR,10bR)-9-Ethoxy-8-methoxy-6-(3-methylsulfinyl-phenyl)-1,2,3,4,4a,10b-hexahydro-phenanthridin-2-ol

Starting Compounds

### A1. Acetic acid (1RS,3RS,4RS)-4-{[1-(4-(toluene-4sulfonylamino)phenyl)methanoyl]amino}-3-(3,4-dimethoxyphenyl)cyclohexyl ester

874 mg of 4-(toluene-4-sulfonylamino)-benzoic acid, 575 mg of N-ethyl-N′-(3-dimethylaminopropyl)carbodiimide hydrochloride (EDCl) and 2 mg of 4-dimethylaminopyridine are placed in a flask under nitrogen. 734 mg of acetic acid (1RS,3RS,4RS)-4-amino-3-(3,4-dimethoxyphenyl)cyclohexyl ester (compound B1) dissolved in 2.5 ml of dichloromethane are added and the solution stirred for 3 h. The reaction is quenched with 3 ml of water. After phase separation the organic layer is washed with saturated NaHCO₃ solution and the water layer reextracted with dichloromethane. After drying the organic layer with magnesium sulfate the residue is purified by chromatography to give 1.198 g of the title compound.

Starting from the appropriate amino starting compounds mentioned below or obtainable in a manner analogous as described below and the appropriate art-known benzoic acid derivatives, further relevant starting compounds can be obtained according to Example A1 or analogously or similarly thereto.

### B1. Acetic acid (1RS,3RS,4RS)-4-amino-3-(3,4-dimethoxyphenyl)cyclohexyl ester

A solution of 10.37 g of acetic acid (1RS,3RS,4RS)-3-(3,4-dimethoxyphenyl)-4-nitrocyclohexyl ester (compound C1) in 240 ml of ethanol is added to a zinc-copper couple, prepared from 16.8 g of zinc powder and 920 mg of copper (II) acetate monohydrate in acetic acid, the resulting suspension is refluxed and treated with 26 ml of acetic acid, 3.2 ml of water and 26 ml of ethanol. The resulting mixture is refluxed for further 15 min. The precipitate is filtered off with suction and the solvent is removed. Chromatographical purification on silica gel using a mixture of petroleum ether/ethyl acetate/triethylamine in the ratio 2/7/1 and concentration of the corresponding eluate fractions afford 5.13 g (55% of theory) of the title compound as a pale brown oil.

R_{f}=0.35 (petroleum ether/ethyl acetate/triethylamine=2/7/1)

Starting from the appropriate starting compounds obtainable for a person skilled in the art in analogy to the exemplary starting compounds and synthesis routes disclosed and described in the present examples, the following compounds B2 to B5 are accesible as described in Example B1.

### B2. Acetic acid (1RS,3RS,4RS)-4-amino-3-(3-ethoxy-4-methoxy-phenyl)-cyclohexyl ester

Starting from compound C2 mentioned below, the title compound is obtained analogously to the procedure as in Example B1.

EF: C₁₇H₂₅NO₄; MW: 307.39

MS: 308.0 (MH⁺)

### B2a. Acetic acid (1R,3R,4R)-4-amino-3-(3-ethoxy-4-methoxy-phenyl)-cyclohexyl ester

24.0 g (55.0 mmol) of the pyroglutamate of the title compound (compound B2b) are suspended in 150 ml of water, 100 ml of dichloromethane are added, then saturated KHCO₃-solution until the gas evolution ceased. After phase separation, reextraction of the water layer and drying the combined organic layers with sodium sulfate the solvent is removed to give 16.9 g of the salt-free title compound.

Analytical Column Chromatography (CHIRALPAK AD-H 250×4.6 mm 5 μ No.ADH0CE-DB030, Eluent: n-Hexan/iPrOH=80/20 (v/v)+0.1% Diethylamine): Retention Time: 6.54 min

### B2b. Acetic acid (1R,3R,4R)-4-amino-3-(3-ethoxy-4-methoxy-phenyl)-cyclohexyl ester, salt with L-pyroglutamic acid

Solution A: 55.2 g (180 mmol) of racemic acetic acid (1RS,3RS,4RS)-4-amino-3-3-ethoxy-4-methoxy-phenyl)-cyclohexyl ester (compound B2) are dissolved in 540 ml of isopropyl acetate.

Solution B: 18.6 g (144 mmol) of L-pyroglutamic acid are dissolved in 260 ml of isopropanol under heating, then 290 ml of isopropyl acetate is added carefully.

Solution B is added to solution A and left for 48 hours. The solid is filtered off and washed with a little isopropyl acetate to give after drying 32.48 g colorless crystals with a ratio of the enantiomers of 97:3 in favour of the title compound.

M.p.: 165-167° C.

B3. Acetic acid (1RS,3RS,4RS)-4-amino-3-[4-(1,1-difluoro-methoxy)-3-methoxy-phenyl]-cyclohexyl ester

Starting from compound C3 mentioned below, the title compound is obtained analogously to the procedure as in Example B1.

EF: C₁₆H₂₁F₂NO₄; MW: 329.35

MS: 330.0 (MH⁺)

B4. Acetic acid (1RS,3RS,4RS)-4-amino-3-[3-(1,1-difluoro-methoxy)-4-methoxy-phenyl]-cyclohexyl ester

Starting from compound C4 mentioned below, the title compound is obtained analogously to the procedure as in Example B1.

EF: C₁₆H₂₁F₂NO₄; MW: 329.35

MS: 330.0 (MH⁺)

### B5. Acetic acid (1RS,3RS,4RS)-4-amino-3-[3-(2,2-difluoro-ethoxy)-4-methoxy-phenyl]-cyclohexyl ester

Starting from compound C5 mentioned below, the title compound is obtained analogously to the procedure as in Example B1.

### B5a. Acetic acid (1R,3R,4R)-4-amino-3-[3-(2,2-difluoro-ethoxy)-4-methoxy-phenyl]-cyclohexyl ester

The title compound is obtained from its pyroglutamate salt (compound B5b) analogously as described for compound B2a using sodium hydrogencarbonate solution.

### B5b. Acetic acid (1R,3R,4R)-4-amino-3-[3-(2,2-difluoro-ethoxy)-4-methoxy-phenyl]-cyclohexyl ester, salt with L-pyroglutamic acid

343 mg (1.00 mmol) of acetic acid (1RS,3RS,4RS)-4-amino-3-[3-2,2-difluoro-ethoxy)-4-methoxy-phenyl]-cyclohexyl ester (compound B5) are dissolved in 3 ml of isopropanol. A solution of 103 mg (0.80 mmol) of L-pyroglutamic acid in 2 ml of isopropanol is added. After filtering and drying 162 mg of the pyroglutamate are isolated with an enantiomeric ratio of 97:3 in favour of the title compound.

### B6. Acetic acid (1SR,3RS,4RS)-3-amino-4-(3-ethoxy-4-methoxy-phenyl)-cyclohexyl ester

3.0 g (7.36 mmol) of acetic acid (1SR,3RS,4RS)-3-tert-butoxycarbonylamino-4-(3-ethoxy-4-methoxy-phenyl)-cyclohexyl ester (compound C6) are dissolved in 6 ml of 4 M HCl in dioxane and stirred for 30 min. After removal of the solvent the residue is dissolved in dichloromethane and 25 ml of sat. NaHCO₃ solution are added carefully. After phase separation, reextraction of the water layer and drying of the combined organic layers (Na₂SO₄) the solvent is removed to give 2.25 g of the title compound.

EF: C17 H25 N O4; MW: 307.39

MS: 308.1 (MH⁺)

### B7. Acetic acid (1SR,3RS,4RS)-3-amino-4-(3,4-dimethoxy-phenyl)-cyclohexyl ester

The title compound can be obtained from compound C7 analogously as described for compound B6.

### C1. Acetic acid (1RS,3RS,4RS)-3-(3,4-dimethoxyphenyl)-4-nitrocyclohexyl ester

10.18 g of (1RS,3RS,4RS)-3-(3,4-dimethoxyphenyl)-4-nitrocyclohexanol (compound D1) are dissolved in 100 ml of acetic anhydride and the solution is heated to 100° C. for 1-2 h. After removal of the solvent, the residue is chromatographed on silica gel using a mixture of petroleum ether/ethyl acetate in the ratio 2/1. Concentration of the corresponding eluate fractions furnish 10.37 g (89% of theory) of the title compound as an oil.

R_{f}=0.32 (petroleum ether/ethyl acetate=2/1)

### C2. Acetic acid (1RS,3RS,4RS)-3-(3-ethoxy-4-methoxy-phenyl)-4-nitrocyclohexyl ester

Starting from compound D9 mentioned below, the title compound is obtained according to the procedure as in Example C1.

Starting from the starting compounds mentioned below, the following are obtained according to the procedure as in Example C1:

### C3. Acetic acid (1RS,3RS,4RS)-3-[4-(1,1-difluoro-methoxy)-3-methoxy-phenyl]-4-nitrocyclohexyl ester

### C4. Acetic acid (1RS,3RS,4RS)-3-[3-(1,1-difluoro-methoxy)-4-methoxy-phenyl]-4-nitrocyclohexyl ester

### C5. Acetic acid (1RS,3RS,4RS)-3-(3-(2,2-difluoro-ethoxy)-4-methoxy-phenyl]-4-nitrocyclohexyl ester

### C6. Acetic acid (1SR,3RS,4RS)-3-tert-butoxycarbonylamino-4-(3-ethoxy-4-methoxy-phenyl)-cyclohexyl ester

22.64 g (65 mmol) of [(1RS,6RS)-6(3-ethoxy-4-methoxy-phenyl)-cyclohex-3enyl]-carbamic acid tert-butyl ester (compound D6) are dissolved in 180 ml of THF and 50 ml of BH₃ (1 M solution in THF) are added dropwise (30 min). After stirring for 2 h the mixture is cooled using an ice bath and a mixture of 30 ml of H₂O₂ (30%) and 60 ml of aqueous NaOH (3 M) is added. The mixture is stirred for 30 min at room temperature. 400 ml of water and 200 ml of dichloromethane are added. After phase separation, reextraction of the water layer and drying of the combined organic layers (Na₂SO₄) the solvent is removed and the crude product (23.42 g, mixture of the two mentioned regioisomers˜2:1 in favour of the title compound) is used directly without further purification.

The crude material from above then is dissolved in 50 ml of pyridine. 50 mg of 4-dimethylaminopyridine and 60 ml of acetic anhydride are added and the mixture stirred for 90 min at 100° C. The solvents and the acetic anhydride are removed (sat. NaHCO₃ solution). Purification by means of chromatography yields 9.4 g of the title compound as colorless foam.

EF: C22 H33 N O6; MW: 407.51

MS: 308.1 (MH⁺-Boc), 407.8 (MH⁺), 430.1 (MNa⁺)

### C7. Acetic acid (1SR,3RS,4RS)-3-tert-butoxycarbonylamino-4-(3,4-dimethoxy-phenyl)-cyclohexyl ester

The title compound can be obtained from compound D7 analogously as described for compound C6.

### D1. (1RS,3RS,4RS)-3-(3,4-Dimethoxyphenyl)-4-nitrocyclohexanol

10 g of (1RS,3RS,4SR)-3-(3,4-dimethoxyphenyl)-4-nitrocyclohexanol (compound E1) are dissolved in 170 ml of absolute 1,2-dimethoxyethane. 14.3 ml of a 30% solution of sodium methanolate in methanol are added dropwise. After complete addition, stirring is continued for 10 min and a mixture consisting of 85% phosphoric acid and methanol is added to pH 1. By adding of saturated potassium hydrogencarbonate solution the resulting suspension is neutralized. The mixture is diluted with water and dichloromethane, the organic layer is separated and extracted with dichloromethane. The solvents are removed under reduced pressure to yield the title compound as a pale yellow oil, which crystallizes. The title compound is used without further purification in the next step.

R_{f}=0.29 (petroleum ether/ethyl acetate=1/1)

M.p.: 126-127° C.

### D2. (1RS,3RS,4RS)-3-(3-Ethoxy-4-methoxy-phenyl)-4-nitrocyclohexanol

Starting from compound E2 mentioned below, the title compound is obtained according to the procedure as in Example D1.

Starting from the appropriate starting compounds mentioned below, the following are obtained according to the procedure as in Example D1:

### D3. (1RS,3RS,4RS)-3-[4-(1,1-Difluoro-methoxy)-3-methoxy-phenyl]-4-nitrocyclohexanol

### D4. (1RS,3RS,4RS)-3-[3-(1,1-Difluoro-ethoxy)-4-methoxy-phenyl)-4-nitrocyclohexanol

### D5. (1RS,3RS,4RS)-3-(3-(2,2-Difluoro-ethoxy)-4-methoxy-phenyl)-4-nitrocyclohexanol

### D6. [(1RS,6RS)-6-(3-Ethoxy-4-methoxy-phenyl)-cyclohex-3-enyl]-carbamic acid tert-butyl ester

Starting from (1RS,6RS)-6-(3-ethoxy-4-methoxy-phenyl)-cyclohex-3-enylamine (compound E6) the title compound is obtained analogously as described for compound D7.

EF: C20 H29 N O4; MW: 347.46,

MS: 370.1 (MNa⁺)

### D7. [(1RS,6RS)-6-(3,4Dimethoxy-phenyl)-cyclohex-3-enyl]-carbamic acid tert-butyl ester

15.18 g (65.06 mmol) of (±)-cis-6-(3,4-dimethoxyphenyl)-cyclohex-3-enylamine (compound E7) and 14.21 g (65.11 mmol) of Boc₂O are stirred in dichloromethane for 2.5 h, then the solvent is removed and the residue crystallized from ethylacetate/n-heptane to give 19.1 g of the title compound.

EF: C19 H27 N O4; MW: 333.43,

MS: 334.2 (MH⁺)

### E1. (1RS,3RS,4SR)-3-(3,4-Dimethoxyphenyl)-4-nitrocyclohexanol

Under nitrogen atmosphere 16.76 g of (3RS,4SR)-3-(3,4-dimethoxyphenyl)-4-nitrocyclohexanone (compound F1) are dissolved in 300 ml of tetrahydrofurane, the solution is cooled to −78° C., and 75 ml of 1 M solution of potassium tri-sec-butylborohydride in tetrahydrofurane is added dropwise. After stirring for further 1 h, a mixture consisting of 30% hydrogeneperoxide solution and phosphate buffer solution is added. Stirring is continued for further 10 min, the reaction mixture is diluted with 400 ml of ethyl acetate and the aqueous layer is extracted with ethyl acetate, the combined organic phases are concentrated to give a foam, which is purified by chromatography on silica gel using a mixture of petroleum ether/ethyl acetate in the ratio 1/1 to furnish 10.18 g (60% of theory) of the title compound.

EF: C₁₄H₁₉NO₅; MW: 281.31

MS: 299.1 (MNH₄⁺)

R_{f}=0.29 (petroleum ether/ethyl acetate=1/1)

M.p.: 139-141° C.

### E2. (1RS,3RS,4SR)-3-(3-Ethoxy-4-methoxy-phenyl)-4-nitrocyclohexanol

Starting from compound F2 mentioned below, the title compound is obtained according to the procedure as in Example E1.

Starting from the appropriate starting compounds mentioned below, the following are obtained according to the procedure as in Example E1:

### E3. (1RS,3RS,4SR)-3-[4-(1,1-Difluoro-methoxy)-3-methoxy-phenyl]-4-nitrocyclohexanol

### E4. (1RS,3RS,4SR)-3-[3-(1,1-Difluoro-methoxy)-4-methoxy-phenyl]-4-nitrocyclohexanol

### E5 (1RS,3RS,4SR)-3-(3-(2,2-Difluoro-ethoxy)-4-methoxy-phenyl)-4-nitrocyclohexanol

### E6. (1RS,6RS)-6-(3-Ethoxy-4-methoxy-phenyl)-cyclohex-3-enylamine

Starting from 2-ethoxy-1-methoxy-4(1RS,6RS)-6-nitro-cyclohex-3-enyl)-benzene (compound F6) the title compound is obtained analogously as described for compound E7.

### E7. (±)-cis-6-(3,4-Dimethoxyphenyl)-cyclohex-3-enylamine

40 g of (±)-cis-1,2-dimethoxy-4-(2-nitrocyclohex-4-enyl)benzene (compound F7) are dissolved in 400 ml of ethanol and 40 g of zinc powder are added. After heating to boiling temperature, 65 ml of glacial acetic acid are added dropwise. Afterwards, the reaction mixture is filtrated and concentrated. The residue is redissolved in diluted hydrochloric acid and extracted with toluene. The aqueous layer is alkalized using 6 N solution of sodium hydroxide and extracted several times with toluene. The combined organic phases of the alkalic extraction are dried using sodium sulfate and concentrated. The residue is chromatographed on silica gel. 11.5 g of the title compound are obtained.

### F1. (3RS,4SR)-3-(3,4-Dimethoxyphenyl)-4-nitrocyclohexanone

90.0 g of 3,4-dimethoxy-ω-nitrostyrene (compound G1), 90 ml of 2-trimethylsilyloxy-1,3-butadiene and 180 ml of abs. toluene are put in an autoclave, where the mixture is stirred at 140° C. for 2 days and then cooled. After addition of 1000 ml of ethyl acetate, 300 ml of a 2 N solution of hydrochloric acid are dropped under stirring. The phases are separated and the aqueous layer is extracted three times with dichloromethane. The combined organic extracts are washed with saturated sodium hydrogencarbonate solution, dried over magnesium sulfate and the solvents are removed under reduced pressure to give 150 g of the crude title compound. Further purification is carried out by chromatography on silica gel using petroleum ether/ethyl acetate in the ratio 1/1 as eluent to give 81.5 g (67% of theory) of the pure title compound.

EF: C₁₄H₁₇NO₅; MW: 279.30

MS: 279 (M⁺), 297.1 (MNH₄⁺)

R_{f}=0.47 (petroleum ether/ethyl acetate=1/1)

M.p.: 147-148° C.

### F2. (3RS,4SR)-3-(3-Ethoxy-4-methoxy-phenyl)-4-nitrocyclohexanone

Starting from compound G2 mentioned below, the title compound is obtained according to the procedure as in Example F1.

Starting from the appropriate starting compounds mentioned below, the following are obtained according to the procedure as in Example F1:

### F3. (3RS,4SR)-3-[4-(1,1-Difluoro-methoxy)-3-methoxy-phenyl]-4-nitrocyclohexanone

### F4. (3RS,4SR)-3-[3-(1,1-Difluoro-methoxy)-4-methoxy-phenyl]-4-nitrocyclohexanone

### F5. (3RS,4SR)-3-(3-(2,2-Difluoro-ethoxy)-4-methoxy-phenyl)-4-nitrocyclohexanone

### F6. 2-Ethoxy-1-methoxy-4-((1RS,6RS)-6-nitro-cyclohex-3-enyl)-benzene

Starting from 2-ethoxy-1-methoxy((1RS,6SR)-nitro-cyclohex-3-enyl)-benzene (compound G6) the title compound is obtained analogously as described for compound F7.

### F7. (±)-cis-1,2-Dimethoxy-4-(2-nitrocyclohex-4-enyl)benzene

10.0 g of (±)-trans-1,2-dimethoxy-4-(2-nitrocyclohex-4-enyl)benzene (compound G7) and 20.0 g of potassium hydroxide are dissolved in 150 ml of ethanol and 35 ml of dimethylformamide. A solution of 17.5 ml of conc. sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized in ethanol. 8.6 g of the title compound of m.p 82.5-84° C. are obtained.

### G1. 3,4-Dimethoxy-ω-nitrostyrene

207.0 g of 3,4dimethoxybenzaldehyde, 100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C.

Yield: 179.0 g.

### G2. 3-Ethoxy-4-methoxy-ω-nitrostyrene

Starting from art-known starting compounds, the title compound is obtained according to the procedure as in Example G1:

Starting from starting compounds, which are art-known or which can be obtained analogously to art-known compounds or according to art-known procedures (such as e.g. as described in WO 95/01338 or analogously or similarly thereto) the following compounds are obtained according to the procedure as in Example G1:

### G3. 4-(1,1-Difluoro-methoxy)-3-methoxy-ω-nitrostyrene

### G4. 3-(1,1-Difluoro-methoxy)-4-methoxy-ω-nitrostyrene

### G5. 3-(2,2-Difluoro-ethoxy-4)-methoxy-ω-nitrostyrene

The title compound is obtained starting from 3-(2,2-difluoro-ethoxy)-4-methoxy-benzaldehyde (compound H1) according to the procedure as in Example G1.

M.p.: 164-165° C.

### G6. 2-Ethoxy-1-methoxy-4-((1RS,6SR)-6-nitro-cyclohex-3-enyl)-benzene

Starting from 3-ethoxy-4-methoxy-ω-nitrostyrene (compound G2) the title compound is obtained analogously as described for compound G7.

### G7. (±)-trans-1,2-Dimethoxy-4-(2-nitrocyclohex-4-enyl)benzene

50.0 g of 3,4-dimethoxy-ω-nitrostyrene (compound G1), and 1.0 g (9.1 mmol) of hydroquinone are suspened in 200 ml of abs. toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol.

M.p.: 113.5-115.5° C.

### H1. 3-(2,2-Difluoro-ethoxy)-4-methoxy-benzaldehyde

10.04 g of isovanillin and 15.5 g of potassium carbonate are placed in an autoclave. 50 ml of DMF are added as well as 12.44 g of 2-bromo-1,1-difluoroethane. The autoclave is closed and heated at 60° C. for 20 h. Then the solids are filtered off and washed with 120 ml of DMF. About 120 ml of the solvent are distilled off and the residue poured on 200 ml of ice/water, where the product preciptates. After stirring the slurry for 30 minutes the product is filtered off and dried to give 13.69 g of the desired product.

M.p.: 66-68° C.

Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia; as well as for enhancing cognition. Yet in addition, the compounds of the invention are useful in the treatment of diabetes mellitus, leukaemia and osteoporosis.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention also relates to the use of the compounds according to the invention for the production of pharmaceutical compositions for treating disorders which are mediated by phosphodiesterases, in particular PDE4mediated disorders, such as, for example, those mentioned in the specification of this invention or those which are apparent or known to the skilled person.

The invention also relates to the use of the compounds according to the invention for the manufacture of pharmaceutical compositions having PDE4 inhibitory activity.

The invention furthermore relates to pharmaceutical compositions for the treatment and/or prophylaxis of the illnesses mentioned comprising one or more of the compounds according to the invention.

The invention yet furthermore relates to compositions comprising one or more compounds according to this invention and a pharmaceutically acceptable carrier. Said compositions can be used in therapy, such as e.g. for treating, preventing or ameliorating one or more of the abovementioned diseases.

The invention still yet furthermore relates to pharmaceutical compositions according to this invention having PDE, particularly PDE4, inhibitory activity.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, excipients, carriers, vehicles, diluents or adjuvants which are suitable for the desired pharmaceutical formulations on account of his/her expert knowledge. In addition to solvents, gel formers, ointment bases and other active compound excipients, for example anti-oxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patent. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarily between 0.01 and 3 mg per day. The customary dose in the case of systemic therapy (p.o. or i.v.) is between 0.003 and 3 mg/kg per day. In another embodiment, the dose for administration by inhalation is between 0.1 and 3 mg per day, and the dose in the case of systemic therapy (p.o. or i.v.) is between 0.03 and 3 mg/kg per day.

Biological Investigations

The second messenger cyclic AMP (cAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular cAMP concentration and thus to the inhibition of cellular activation (J E Souness et al., Immunopharmacology 47:127-162, 2000).

The antiinflammatory potential of PDE4 inhibitors in vivo in various animal models has been described (M M Teixeira, TiPS 18:164-170,1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factors in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973,1999). Substances which inhibit the secretion of the afore-mentioned proinflammatory mediators are those which inhibit PDE4. PDE4 inhibition by the compounds according to the invention is thus a central indicator for the suppression of inflammatory processes.

Methods for Measuring Inhibition of PDE4 Activity

The PDE4B2 (GB no. M97515) was a gift of Prof. M. Conti (Stanford University, USA). It was amplified from the original plasmid (pCMV5) via PCR with primers Rb9 (5′- GCCAGCGTGCAAATAATGAAGG -3′) and Rb10 (5′-AGAGGGGGATTATGTATCCAC-3′) and cloned into the pCR-Bac vector (Invitrogen, Groningen, N L).

The recombinant baculovirus was prepared by means of homologous recombination in SF9 insect cells. The expression plasmid was cotransfected with Bac-N-Blue (Invitrogen, Groningen, N L) or Baculo-Gold DNA (Pharmingen, Hamburg) using a standard protocol (Pharmingen, Hamburg). Wt virus-free recombinant virus supernatant was selected using plaque assay methods. After that, high-titre virus supernatant was prepared by amplifying 3 times. PDE was expressed in SF21 cells by infecting 2×10⁶ cells/ml with an MOI (multiplicity of infection) between 1 and 10 in serum-free SF900 medium (Life Technologies, Palsley, UK). The cells were cultured at 28° C. for 48-72 hours, after which they were pelleted for 5-1 0 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 100×g and the supernatant was stored at −80° C. until subsequent use (see below). The protein content was determined by the Bradford method (BioRad, Munich) using BSA as the standard.

PDE4B2 activity is inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 1 μl of the respective substance dilution in DMSO and sufficient recombinant PDE (100×g supernatant, see above) to ensure that 10-20% of the cAMP is converted under the said experimental conditions. The final concentration of DMSO in the assay (1% v/v) does not substantially affect the activity of the PDE investigated. After a preincubation of 5 min at 37° C., the reaction is started by adding the substrate (cAMP) and the assay is incubated for a further 15 min; after that, it is stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

Representative inhibitory values determined for the compounds according to the Invention follow from the following table A, in which the numbers of the compounds correspond to the numbers of the Examples.

TABLE A
Inhibition of the PDE4 activity

| Compound | −log IC₅₀ [mol/l] |
|---|---|
| 1 to 4, 6 to 10, 24 to | The inhibitory values of |
| 42, 45, and 61 | these listed compounds are |
| | in the range from 7.52 to |
| | 10.06 |

1. A compound of formula I, 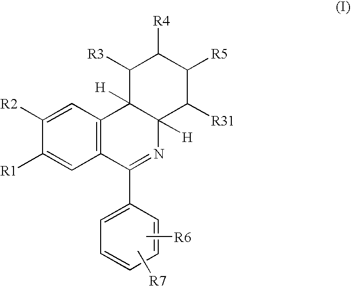
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
or in which
R1 and R2 together are a 1-2C-alkylenedioxy group,
R3 is hydrogen or 1-4C-alkyl,
R31 is hydrogen or 1-4C-alkyl,
wherein either, in a first embodiment (embodiment a),
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
or, in a second embodiment (embodiment b),
R4 is hydrogen or 1-4C-alkyl, and
R5 is —O—R51, in which
R51 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl,
R6 is hydrogen, halogen, 1-4C-alkyl or 1-4C-alkoxy,
wherein in a first aspect (aspect 1),
R7 is —S(O)₂N(R8)R9, in which
R8 is hydrogen, 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
or R8 and R9 together and with inclusion of the nitrogen atom, to which they are attached, form a heterocyclic ring Het1, in which
Het1 is optionally substituted by R81, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R81 is 1-4C-alkyl,
or, in a second aspect (aspect 2),
R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
or, in a third aspect (aspect 3),
R7 is —S(O)ₙR12, in which
n is 0, 1 or 2,
R12 is 1-4C-alkyl,
or an enantiomer, salt, N-oxide, salt of an N-oxide or enantiomer of an N-oxide thereof.

2. A compound of formula I according to claim 1 in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
wherein either, in a, first embodiment (embodiment a),
R4 is —O—R41, in which
R41 is hydrogen or 1-7C-alkylcarbonyl,
R5 is hydrogen,
or, in a second embodiment (embodiment b),
R4 is hydrogen, and
R5 is —O—R51, in which
R51 is hydrogen or 1-7C-alkylcarbonyl,
R6 is hydrogen or 1-4C-alkyl,
wherein in a first aspect (aspect 1),
R7 is —S(O)₂N(R8)R9, in which
R8 is hydrogen, 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
or R8 and R9 together and with inclusion of the nitrogen atom, to which they are attached, form a heterocyclic ring Het1, in which
Het1 is a 3- to 7-membered saturated monocyclic heterocyclic ring radical comprising the nitrogen atom, to which R8 and R9 are bonded, and optionally one further heteroatom selected from the group consisting of oxygen, nitrogen, N(R81) and sulfur, in which
R81 is 1-4C-alkyl,
or, in a second aspect (aspect 2),
R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
or, in a third aspect (aspect 3),
R7 is —S(O)ₙR12, in which
n is 0, 1 or 2,
R12 is 1-4C-alkyl,
or an enantiomer, salt, N-oxide, salt of an N-oxide or enantiomer of an N-oxide thereof.

3. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is 1-4C-alkylcarbonyl or hydrogen,
R5 is hydrogen,
R6 is hydrogen or methyl,
wherein in a first aspect (aspect 1),
R7 is —S(O)₂N(R8)R9, in which
R8 is 1-4C-alkyl, 1-4C-alkoxy-2-4C-alkyl or 3-7C-cycloalkyl,
R9 is hydrogen, 1-4C-alkyl or 1-4C-alkoxy-2-4C-alkyl,
or R8 and R9 together and with inclusion of the nitrogen atom, to which they are attached, form a heterocyclic ring Het1, in which
Het1 is morpholinyl, thiomorpholinyl, pyrrolidinyl, piperidinyl, 4-N—(R81)-piperazinyl, or 4-N—(R81)-homopiperazinyl, in which
R81 is 1-4C-alkyl,
or, in a second aspect (aspect 2),
R7 is -A-N(R10)S(O)₂—R11, in which
A is a bond or 1-4C-alkylene,
R10 is hydrogen or 1-4C-alkyl,
R11 is 1-4C-alkyl, or R111-substituted phenyl, in which
R111 is halogen or 1-4C-alkyl,
or, in a third aspect (aspect 3),
R7 is —S(O)ₙR12, in which
n is 0, 1 or 2,
R12 is 1-4C-alkyl,
or an enantiomer, salt, N-oxide, salt of an N-oxide or enantiomer of an N-oxide thereof.

4. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is acetyl or hydrogen,
R5 is hydrogen,
R6 is hydrogen or methyl,
wherein in a first aspect (aspect 1),
R7 is —S(O)₂N(R8)R9, in which
R8 is 1-4C-alkyl, 1-4C-alkoxy-ethyl or 3-5C-cycloalkyl,
R9 is hydrogen, 1-4C-alkyl or 1-4C-alkoxy-ethyl,
or R8 and R9 together and with inclusion of the nitrogen atom, to which they are attached, form a heterocyclic ring Het1, in which
Het1 is morpholinyl, pyrrolidinyl, piperidinyl or 4-N—(R81)-piperazinyl, in which
R81 is 1-4C-alkyl,
or, in a second aspect (aspect 2),
R7 is -A-N(R10)S(O)₂—R11, in which
A is a bond or 1-2C-alkylene,
R10 is hydrogen or 1-4C-alkyl,
R11 is 1-4C-alkyl, or R111-substituted phenyl, in which
R111 is fluorine, chlorine or 1-4C-alkyl,
or, in a third aspect (aspect 3),
R7 is —S(O)ₙR12, in which
n is 0, 1 or 2,
R12 is 1-4C-alkyl,
or an enantiomer, salt, N-oxide, salt of an N-oxide or enantiomer of an N-oxide thereof.

5. A compound of formula I according to claim 1 in which
R1 is methoxy or ethoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
wherein in a first aspect (aspect 1),
R7 is —S(O)₂N(R8)R9, in which
R8 is methyl, ethyl, propyl, 2-methoxy-ethyl or cyclopropyl,
R9 is hydrogen, methyl, ethyl, propyl or 2-methoxy-ethyl,
or R8 and R9 together and with inclusion of the nitrogen atom, to which they are attached, form a heterocyclic ring Het1, in which
Het1 is morpholinyl, pyrrolidinyl, piperidinyl or 4-N—(R81)-piperazinyl, in which
R81 is methyl,
or, in a second aspect (aspect 2),
R7 is -A-N(R10)S(O)₂—R11, in which
A is a bond or methylene,
R10 is hydrogen or methyl,
R11 is R111-substituted phenyl, in which
R111 is fluorine, chlorine or methyl,
or, in a third aspect (aspect 3),
R7 is —S(O)ₙR12, in which
n is 0, 1 or 2,
R12 is 1-4C-alkyl,
or an enantiomer, salt, N-oxide, salt of an N-oxide or enantiomer of an N-oxide thereof.

6. A compound of formula I according to claim 1 in which
R1 is methoxy or ethoxy,
R2 is methoxy, ethoxy, 2,2-difluoroethoxy, or difluoromethoxy,
R3 is hydrogen,
R31 is hydrogen,
R4 is —O—R41, in which
R41 is hydrogen,
R5 is hydrogen,
R6 is hydrogen,
R7 is —S(O)ₙR12, in which
n is 0 or 1,
R12 is methyl,
or an enantiomer, salt, N-oxide, salt of an N-oxide or enantiomer of an N-oxide thereof.

7. A compound of formula I according to claim 1 comprising one or more of the following:
R1 is methoxy,
R2 is ethoxy, difluoromethoxy or 2,2-difluoroethoxy, and
R3 and R31 are both hydrogen,
R4 is —O—R41, in which
R41 is hydrogen, and
R5 is hydrogen,
R6 is hydrogen and
R7 is bonded to the meta- or para position with respect to the binding position at which the phenyl ring is bonded or an enantiomer, salt, N-oxide, salt of an N-oxide or enantiomer of an N-oxide thereof.

8. A compound of formula I according to claim 1 selected from the group consisting of N-[4-((2RS,4aRS,10bRS)-2-Hydroxy-8,9-dimethoxy-1,2,3,4,4a,10b-hexahydro-phenanthridin-6-yl)-phenyl]-4,N-dimethyl-benzenesulfonamide,
4-Fluoro-N-[4-((2RS,4aRS,10bRS)-2-hydroxy-8,9-dimethoxy-1,2,3,4,4a,10b-hexahydro-phenanthridin-6-yl)-phenyl]-benzenesulfonamide,
N-[4-((2RS,4aRS,10bRS)-2-Hydroxy-8,9-dimethoxy-1,2,3,4,4a,10b-hexahydro-phenanthridin-6-yl)-2-methyl-phenyl]-4-methyl-benzenesulfonamide,
N-[4-((2RS,4aRS,10bRS)-2-Hydroxy-8,9-dimethoxy-1,2,3,4,4a,10b-hexahydro-phenanthridin-6-yl)-benzyl]-4-methyl-benzenesulfonamide,
N-{4-[(2RS,4aRS,10bRS)-8-(1,1-Difluoro-methoxy)-2-hydroxy-9-methoxy-1,2,3,4,4a,10b-hexahydro-phenanthridin-6-yl]-phenyl}-methanesulfonamide,
N-{4-[(2RS,4aRS,10bRS)-8-(1,1-Difluoro-methoxy)-2-hydroxy-9-methoxy-1,2,3,4,4a,10b-hexahydro-phenanthridin-6-yl]-phenyl}-4-methyl-benzenesulfonamide,
N-{4-[(2RS,4aRS,10bRS)-8-(1,1-Difluoro-methoxy)-2-hydroxy-9-methoxy-1,2,3,4,4a,10b-hexahydro-phenanthridin-6-yl]-phenyl}-4-fluoro-benzenesulfonamide,
N-{4-[(2RS,4aRS,10bRS)-8-(1,1-Difluoro-methoxy)-2-hydroxy-9-methoxy-1,2,3,4,4a,10b-hexahydro-phenanthridin-6-yl]-benzyl}-4-methyl-benzenesulfonamide,
N-{4-[(2RS,4aRS,10bRS)-9-(1,1-Difluoro-methoxy)-2-hydroxy-8-methoxy-1,2,3,4,4a,10b-hexahydro-phenanthridin-6-yl]-phenyl}-4-methyl-benzenesulfonamide,
N-{4-[(2RS,4aRS,10bRS)-9-(1,1-Difluoro-methoxy)-2-hydroxy-8-methoxy-1,2,3,4,4a,10b-hexahydro-phenanthridin-6-yl]-benzyl}-4-methyl-benzenesulfonamide,
N-[4-((2RS,4aRS,10bRS)-2-Hydroxy-8,9-dimethoxy-1,2,3,4,4a,10b-hexahydro-phenanthridin-6-yl)-phenyl]-4-methyl-benzenesulfonamide,
4-((2RS,4aRS,10bRS)-2-Hydroxy-8,9-dimethoxy-1,2,3,4,4a,10b-hexahydro-phenanthridin-6-yl)-N,N-dipropyl-benzenesulfonamide,
4-((2RS,4aRS,10bRS)-9-Ethoxy-2-hydroxy-8-methoxy-1,2,3,4,4a,10b-hexahydro-phenanthridin-6-yl)-N,N-dipropyl-benzenesulfonamide,
4-((2RS,4aRS,10bRS)-9-Ethoxy-2-hydroxy-8-methoxy-1,2,3,4,4a,10b-hexahydro-phenanthridin-6-yl)-N-(2-methoxy-ethyl)-N-methyl-benzenesulfonamide,
(2RS,4aRS,10bRS)-8-(1,1-Difluoro-methoxy)-9-methoxy-6-[4-(pyrrolidine-1-sulfonyl)-phenyl]-1,2,3,4,4a,10b-hexahydro-phenanthridin-2-ol,
(2RS,4aRS,10bRS)-8-(1,1-Difluoro-methoxy)-9-methoxy-6-[4-(piperidine-1-sulfonyl)-phenyl]-1,2,3,4,4a,10b-hexahydro-phenanthridin-2-ol,
(2RS,4aRS,10bRS)-8-(1,1-Difluoro-methoxy)-9-methoxy-6-[3-(pyrrolidine-1-sulfonyl)-phenyl]-1,2,3,4,4a,10b-hexahydro-phenanthridin-2-ol,
4-[(2RS,4aRS,10bRS)-8-(1,1-Difluoro-methoxy)-2-hydroxy-9-methoxy-1,2,3,4,4a,10b-hexahydro-phenanthridin-6-yl]-N,N-dipropyl-benzenesulfonamide,
(2RS,4aRS,10bRS)-8-(1,1-Difluoro-methoxy)-9-methoxy-6-[4-(morpholine-4-sulfonyl)-phenyl]-1,2,3,4,4a,10b-hexahydro-phenanthridin-2-ol,
(2RS,4aRS,10bRS)-8-(1,1-Difluoro-methoxy)-9-methoxy-6-[3-(morpholine-4-sulfonyl)-phenyl]-1,2,3,4,4a,10b-hexahydro-phenanthridin-2-ol,
(2RS,4aRS,10bRS)-8-(1,1-Difluoro-methoxy)-9-methoxy-6-[3-(piperidine-1-sulfonyl)-phenyl]-1,2,3,4,4a,10b-hexahydro-phenanthridin-2-ol,
3-[(2RS,4aRS,10bRS)-8-(1,1-Difluoro-methoxy)-2-hydroxy-9-methoxy-1,2,3,4,4a,10b-hexahydro-phenanthridin-6-yl]-N,N-dimethyl-benzenesulfonamide,
N-Cyclopropyl-3-[(2RS,4aRS,10bRs)-8-(1,1-difluoro-methoxy)-2-hydroxy-9-methoxy-1,2,3,4,4a,10b-hexahydro-phenanthridin-6-yl]-benzenesulfonamide,
3-[(2RS,4aRS,10bRS)-8-(1,1-Difluoro-methoxy)-2-hydroxy-9-methoxy-1,2,3,4,4a,10b-hexahydro-phenanthridin-6-yl]-N,N-bis-(2-methoxy-ethyl)-benzenesulfonamide,
(2RS,4aRS,10bRS)-8-(1,1-Difluoro-methoxy)-9-methoxy-6-[3-(4-methyl-piperazine-1-sulfonyl)-phenyl]-1,2,3,4,4a,10b-hexahydro-phenanthridin-2-ol,
(2RS,4aRS,10bRS)-9-(2,2-Difluoro-ethoxy)-8-methoxy-6-[3-(4-methyl-piperazine-1-sulfonyl)-phenyl]-1,2,3,4,4a,10b-hexahydro-phenanthridin-2-ol,
(2RS,4aRS,10bRS)-9-(2,2-Difluoro-ethoxy)-8-methoxy-6-[4-(pyrrolidine-1-sulfonyl)-phenyl]-1,2,3,4,4a,10b-hexahydro-phenanthridin-2-ol,
(2RS,4aRS,10bRS)-6-(3-Methanesulfonyl-phenyl)-8,9-dimethoxy-1,2,3,4,4a,10b-hexahydro-phenanthridin-2-ol,
(2RS,4aRS,10bRS)-9-Ethoxy-8-methoxy-6-(4-methylsulfanyl-phenyl)-1,2,3,4,4a,10b-hexahydro-phenanthridin-2-ol,
(2R,4aR,10bR)-9-Ethoxy-8-methoxy-6-(4-methylsulfanyl-phenyl)-1,2,3,4,4a,10b-hexahydro-phenanthridin-2-ol, (2RS,4aRS,10bRS)-9-(2,2-Difluoro-ethoxy)-8-methoxy-6-(4-methylsulfanyl-phenyl)-1,2,3,4,4a,10b-hexahydro-phenanthridin-2-ol,
(2RS,4aRS,10bRS)-9-(2,2-Difluoro-ethoxy)-8-methoxy-6-(3-methylsulfanyl-phenyl)-1,2,3,4,4a,10b-hexahydro-phenanthridin-2-ol,
(2RS,4aRS,10bRS)-9-(2,2-Difluoro-ethoxy)-8-methoxy-6-(3-methylsulfinyl-phenyl)-1,2,3,4,4a,10b-hexahydro-phenanthridin-2-ol,
(2R,4aR,10bR)-9-Ethoxy-8-methoxy-6-(4-methylsulfinyl-phenyl)-1,2,3,4,4a,10b-hexahydro-phenanthridin-2-ol, and the enantiomers, salts, N-oxides, salts of the N-oxides and enantiomers of the N-oxides thereof.

9. A compound of formula I according to claim 1, which has with respect to the positions 4a and 10b the configuration shown in formula I*: 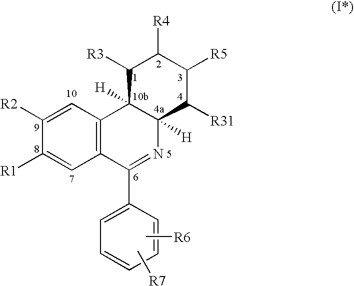
or a salt, N-oxide or salt of an N-oxide thereof.

10. Compounds A compound of formula I according to claim 1, which has with respect to the positions 2, 4a and 10b the configuration shown in formula Ia*****, or, which has with respect to the positions 3, 4a and 10b the configuration shown in formula Ib*****: 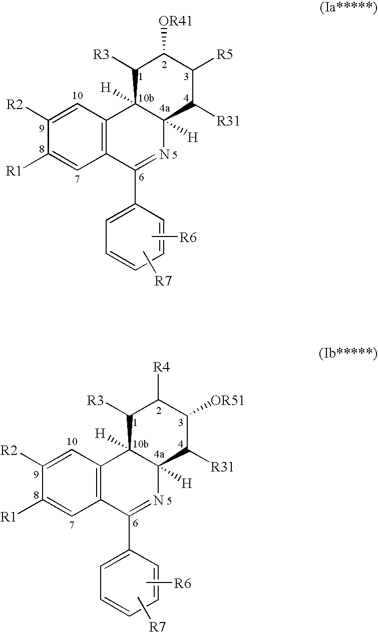
or a salt, N-oxide or salt of an N-oxide thereof.

11. (canceled)

12. A pharmaceutical composition comprising one or more compounds of formula I as claimed in claim 1, or a pharmaceutically acceptable enantiomer, salt, N-oxide, salt of an N-oxide or enantiomer of an N-oxide thereof, together with a pharmaceutically acceptable excipient and/or vehicle.

13-14. (canceled)

15. A method for treating an illness in a patient comprising administering to said patient a therapeutically effective amount of a compound of formula I as claimed in claim 1, or a pharmaceutically acceptable enantiomer, salt, N-oxide, salt of an N-oxide or enantiomer of an N-oxide thereof.

16. A method for treating an airway disorder in a patient comprising administering to said patient a therapeutically effective amount of a compound of formula I as claimed in claim 1, or a pharmaceutically acceptable enantiomer, salt, N-oxide, salt of an N-oxide or enantiomer of an N-oxide thereof.

17. A method for treating a PDE-mediated disorder in a patient comprising administering to said patient a therapeutically effective amount of a compound of formula I as claimed in claim 1, or a pharmaceutically acceptable enantiomer, salt, N-oxide, salt of an N-oxide or enantiomer of an N-oxide thereof.
